SEQUENCE LISTING (18 sequences)

SEQ ID NO: 1 - DNA, 1836 bp, Mycobacterium avium
  CDS (1)..(1836): MAP3694 gene encoding MAP3694 polypeptide
atgagccactacaagagcaacgtccgcgaccaggaattcaacctgttcgaagtgctgggcgttgacaaggctttagggcaaggcgagttcagcgatctggacgccgacacggcccgcgagatgttgaacgaaatcagccggctggccgaggggcccatcgccgactccttcgtcgagggcgaccgcaacccgccggtgttcgacccgaagacgcactccgtgacgctgccggaatcgttcaagaagtccgtccacgccgtcatcgaggccgggtgggacaaggtaggcatcgacgaggccctcggcggggtcgccatgcccaaggcgctgctgtgggcgctgcacgagcacatcctgggcgccaacccggcggtctggatgtacgccggcggcgcgggcttcgccaacatcctctaccacctgggcaccgacgagcagaagaagtgggccgtgatggccgccgagcgcggctggggttcgaccatggtgctcaccgagccggacgcgggttccgacgtcggcgccgggcgcaccaaggcggtcaagcaggacgacggctcctggcacatcgacggtgtgaagcgattcatcacctcggccgactccgacgacctgttcgagaacatcttccacctggtgctggcccgccccgagggcgccggcccgggcaccaagggcctgtcgctgttcttcgtgcccaagttcctgttcgacttcgagaccggtgagctcggcgagcgcaacggcgtcttcgtcaccaatgtcgagcacaagatgggcctgaaggtgtcggcgacctgcgagctgagcttcggccagcacgacgtcccggccaagggctggctggtcggcgaggtgcacaacgggatcgcgcagatgttcgaggtcatcgagcaggcccgcatgatggtgggcaccaaggccatcgcgaccctgtcgaccggttacctgaacgcgttggagtacgccaagtctcgcgtccagggcgccgacatgacccagatgaccgacaagaccgcaccgcgggtgaccatcacgcaccacccggacgtgcggcggtcgctgatgacccagaaggcctacgccgagggtctgcgcgcgctgtacctgttcacctcgacctaccaggacgcggcggtcgccgaggcgctctacggggtggacgccgaactggcggtcaaggtcaacgacctgatgctgccggtggtcaagggcgtcggctccgagcaggcctacgccaagctcaccgagagcctgcagaccttcggtggctccggcttcctgcaggactacccgattgagcagtacatccgcgactccaagatcgactcgctctacgagggcaccacggccatccaggcgcaggacttcttcttccgcaagatcgtccgcgacaagggtgtggcgctggcccacgtgtccgagcagatccagaagttcgtcgacagcgagtccggcaacggccggctcaagtccgagcgcgaactgctggccaaggcgctggcggacgtgcagggcatggcgaccgcgctgaccggctacctgatggccgcccaggaggacgtgaccagcctgtacaaggtgggcctgggttcggtgcggttcctgatgagcgtcggtgacctgatcatcggctggttgctgcagcgtcaggcggcggtggccgtggcggcgctggacgccggcgccagcggggaggaccggtccttctacgagggcaagatcgcggtggcgtcgttcttcgcgaagaacttcctgccgatgctgaccagcacccgcgaggtcatcgagacgctggacaacgacatcatggagctcgacgaggcggccttctaa

SEQ ID NO: 2 - protein, 611 aa, Mycobacterium avium
MSHYKSNVRDQEFNLFEVLGVDKALGQGEFSDLDADTAREMLNEISRLAEGPIADSFVEGDRNPPVFDPKTHSVTLPESFKKSVHAVIEAGWDKVGIDEALGGVAMPKALLWALHEHILGANPAVWMYAGGAGFANILYHLGTDEQKKWAVMAAERGWGSTMVLTEPDAGSDVGAGRTKAVKQDDGSWHIDGVKRFITSADSDDLFENIFHLVLARPEGAGPGTKGLSLFFVPKFLFDFETGELGERNGVFVTNVEHKMGLKVSATCELSFGQHDVPAKGWLVGEVHNGIAQMFEVIEQARMMVGTKAIATLSTGYLNALEYAKSRVQGADMTQMTDKTAPRVTITHHPDVRRSLMTQKAYAEGLRALYLFTSTYQDAAVAEALYGVDAELAVKVNDLMLPVVKGVGSEQAYAKLTESLQTFGGSGFLQDYPIEQYIRDSKIDSLYEGTTAIQAQDFFFRKIVRDKGVALAHVSEQIQKFVDSESGNGRLKSERELLAKALADVQGMATALTGYLMAAQEDVTSLYKVGLGSVRFLMSVGDLIIGWLLQRQAAVAVAALDAGASGEDRSFYEGKIAVASFFAKNFLPMLTSTREVIETLDNDIMELDEAAF

SEQ ID NO: 3 - DNA, 300 bp, Mycobacterium avium
  CDS (1)..(300): MAP1507 gene encoding MAP1507 polypeptide
atgtcgttcgtgaccacacagccggaggctctggccgcggcggccgggaacctgcaggctatcgggtggaccttgagcgcccagaacgcggccgctgcagccccaacgacgggggtggtacccgccgctgccgacgaggtctcggcgctgaccgcagcccagttcgccgcgtacgcgcagatgtaccaggcagtgagcgcccaggccgccgccatccacgaggcgttcgtcaacaccctgtcgaccagctccgtttcctacgcggcaaccgaagcagccaacgccgccgcggccctctga

SEQ ID NO: 4 - protein, 99 aa, Mycobacterium avium
MSFVTTQPEALAAAAGNLQAIGWTLSAQNAAAAAPTTGVVPAAADEVSALTAAQFAAYAQMYQAVSAQAAAIHEAFVNTLSTSSVSYAATEAANAAAAL

SEQ ID NO: 5 - DNA, 297 bp, Mycobacterium avium
  CDS (1)..(297): MAP1508 gene encoding MAP1508 polypeptide
atggcaacacgttttatgactgacccgcacgaaatgcgggcgatggcgggccgcttcgaggtgcacgcccagaccgtggaggacgaggcccgcaagatgtgggcgtcgtcgatgaacatcgccggtgcgggctggagtggtcaggcccaggcgacctcgtacgacacgatgggtcaggtcaaccaggccttccgcaacatcgtcaacatgctgcacggggtgcgtgacgggctgatccgcgacgccaacaactacgagcagcaagagcaggcttcgcagcagatcctcagcagctag

SEQ ID NO: 6 - protein, 98 aa, Mycobacterium avium
MATRFMTDPHEMRAMAGRFEVHAQTVEDEARKMWASSMNIAGAGWSGQAQATSYDTMGQVNQAFRNIVNMLHGVRDGLIRDANNYEQQEQASQQILSS

SEQ ID NO: 7 - DNA, 294 bp, Mycobacterium avium
  CDS (1)..(294): MAP3783 gene encoding MAP3783 polypeptide
atgagcatgttggacgctcacattccgcagttggtagccgcacaatcggcgttcagcgccaaggcggcgctgatgcgcagcacgatcagccaggccgagcaggaagcggtctcggcccaggcgttccaccagggcgagtcctcggcggcattccaggccgcgcacgcccgcttcgtggaagtcgccgcccgggtcaacaccctgctggacatcgcccaggccaacctcggcgacgccgccggcacctacgtggccgccgacgccgccgccgcgtccggctacaccgcgttctga

SEQ ID NO: 8 - protein, 97 aa, Mycobacterium avium
MSMLDAHIPQLVAAQSAFSAKAALMRSTISQAEQEAVSAQAFHQGESSAAFQAAHARFVEVAARVNTLLDIAQANLGDAAGTYVAADAAAASGYTAF

SEQ ID NO: 9 - DNA, 291 bp, Mycobacterium avium
  CDS (1)..(291): MAP3784 gene encoding MAP3784 polypeptide
atgtcacagatcatgtacaactacccggcgatgctgagccacgccgccgacatgtcgggctatgccggcacgatgcagggcctgggcgccgacatcgccagcgagcaggccaccctgtccaacgcctggcagggtgacaccgggatgacctaccaggtgtggcaggcgcagtggaacgaggccatggagagcctggtgcgcgcctaccagtcgatggccagcacccacgaggccaacaccatgtccatgttggcccgcgaccaggccgaagccgccaaatggggcggctag

SEQ ID NO: 10 - protein, 96 aa, Mycobacterium avium
MSQIMYNYPAMLSHAADMSGYAGTMQGLGADIASEQATLSNAWQGDTGMTYQVWQAQWNEAMESLVRAYQSMASTHEANTMSMLARDQAEAAKWGG

SEQ ID NO: 11 - DNA, 1266 bp, Mycobacterium avium
  CDS (1)..(1266): MAP fusion gene encoding MAP1507-MAP1508- MAP3783-MAP3784 fusion polypeptide Residues 2-7 His tag; residues 1,8-105 MAP1507; 114-211 MAP1508; 220-316MAP3783; 326-421 MAP3784. Residues 106-113/212-219/317-325 spacers
atgcaccaccaccaccatcattcgttcgtcaccacccagccagaggcgctggctgcggcagcgggtaatctgcaggcaattggctggacgttgagcgcgcaaaatgctgcggctgcagcgccgaccacgggtgtggtcccggcagcggcggacgaggtttctgcgctgaccgccgcacagtttgctgcgtacgcgcagatgtatcaagcagttagcgcgcaagcagccgcaatccacgaagcctttgttaacaccctgagcaccagcagcgtcagctacgctgcgaccgaggcggccaatgcggcagccgccctgtctgcctgctactgtgaactgagcatggcgacgcgcttcatgactgatccgcacgagatgcgtgcgatggcgggtcgcttcgaagtccacgcccagacggtggaggacgaggcccgcaagatgtgggcttccagcatgaacattgcgggtgcgggttggagcggccaggcgcaggcaactagctacgacaccatgggtcaggtaaatcaagcgttccgcaatatcgtcaacatgctgcacggcgttcgtgacggtctgattcgtgatgcaaacaactatgaacaacaagagcaagcatctcaacagatcctgtccagcggtggtagcggtggcggtagcggcatgagcatgttggatgcccacatcccgcaactggtggctgcgcagagcgcgtttagcgcgaaagcggcgttgatgcgtagcaccattagccaggcggagcaggaagcagtgtccgcacaggcattccatcaaggtgagagcagcgcggcattccaagcggcgcatgcccgttttgttgaagtggcagcccgcgtgaacaccctgctggatattgcacaggcgaatttgggcgacgcggcaggtacgtatgtggcggcagatgcggctgcggcgtccggttacacggcttttggtctggttccgcgtggctctaccggtatgagccaaatcatgtacaattatccggcgatgctgagccatgccgcggacatgagcggctacgccggtacgatgcaaggcctgggtgcagatatcgcaagcgaacaggcgacgctgtccaacgcctggcagggcgacaccggcatgacctatcaagtctggcaggcgcagtggaatgaagcaatggagtcgctggttcgtgcctaccagagcatggctagcacgcatgaggccaacaccatgtccatgctggcacgtgaccaagcggaagccgcgaaatggggcggttaa

SEQ ID NO: 12 - protein, 421 aa, Mycobacterium avium
MHHHHHHSFVTTQPEALAAAAGNLQAIGWTLSAQNAAAAAPTTGVVPAAADEVSALTAAQFAAYAQMYQAVSAQAAAIHEAFVNTLSTSSVSYAATEAANAAAALSACYCELSMATRFMTDPHEMRAMAGRFEVHAQTVEDEARKMWASSMNIAGAGWSGQAQATSYDTMGQVNQAFRNIVNMLHGVRDGLIRDANNYEQQEQASQQILSSGGSGGGSGMSMLDAHIPQLVAAQSAFSAKAALMRSTISQAEQEAVSAQAFHQGESSAAFQAAHARFVEVAARVNTLLDIAQANLGDAAGTYVAADAAAASGYTAFGLVPRGSTGMSQIMYNYPAMLSHAADMSGYAGTMQGLGADIASEQATLSNAWQGDTGMTYQVWQAQWNEAMESLVRAYQSMASTHEANTMSMLARDQAEAAKWGG

SEQ ID NO: 13 - DNA, 1854 bp, Mycobacterium avium
  CDS (1)..(1854): rMAP3694 gene encoding His-tagged MAP3694 polypeptide
atgcatcaccaccaccatcacagccactacaaaagcaacgtccgtgatcaggagttcaacttgtttgaggttctgggtgtggacaaagctctgggtcagggtgagtttagcgacctggacgctgatacggcacgcgagatgttgaatgaaatttcccgtctggcggagggcccgattgcagatagcttcgtcgagggtgaccgtaatccgccggtttttgacccgaaaacccatagcgtcacgctgccggaaagctttaagaaatcggtgcatgccgtgattgaggcaggttgggacaaggtgggtatcgatgaggcgttgggtggcgttgcaatgccaaaagctctgctgtgggccctgcatgaacacatcctgggcgccaacccggcagtctggatgtacgccggtggcgcaggctttgctaacatcctgtaccatctgggtaccgatgagcaaaagaagtgggctgttatggcggctgaacgcggctggggcagcaccatggttctgaccgagccggatgcgggcagcgacgtgggcgcaggtcgtacgaaagccgtgaaacaagatgacggttcttggcacatcgatggtgttaaacgtttcatcaccagcgccgattccgacgacctgttcgagaatatcttccatctggtcctggcacgccctgagggtgcgggtccgggcacgaaaggcctgtcgctgtttttcgttccgaagtttctgttcgattttgaaacgggcgaactgggtgagcgcaacggtgtgtttgttacgaatgtcgaacacaaaatgggtctgaaagtgagcgcgacctgcgagttgagcttcggccaacacgacgttccggcgaaaggctggctggtcggtgaagtgcacaacggtatcgcgcagatgttcgaggttatcgagcaagcacgtatgatggtgggtacgaaggcgattgctaccctgtccaccggctatttgaatgccctggaatatgcgaagtcccgtgtgcaaggcgcagacatgacccagatgaccgacaaaaccgcaccgcgtgtaaccatcacccatcacccggatgtccgccgtagcttgatgacgcagaaggcgtacgcagagggcctgcgtgcgctgtacctgttcacctccacctaccaggatgcggcggttgcggaagcgttgtatggcgtcgatgcggaactggcggttaaggttaatgacctgatgctgccggtggtgaaaggtgtcggcagcgagcaggcctatgcaaagctgaccgagagcctgcaaacctttggtggcagcggttttctgcaggattacccgattgaacagtatattcgtgactctaaaatcgatagcctgtacgaaggtacgacggcgattcaggcccaggactttttctttcgcaagattgtgcgtgacaagggcgtcgcgttggcgcacgtgagcgagcaaattcagaaattcgtggacagcgaaagcggtaacggtcgtctgaagagcgaacgcgagctgctggcaaaagccctggctgacgtgcaaggtatggcgactgcgctgacgggctacctgatggctgcgcaagaggatgttactagcctgtataaggtcggtctgggtagcgtccgcttcctgatgtccgttggtgacttgatcattggttggctgctgcagcgtcaagccgcagtcgcggttgccgctttggatgcgggtgcgagcggtgaagatcgctctttctatgagggtaagattgcagttgcgagcttctttgcgaagaacttcctgccaatgctgaccagcacccgtgaagtgattgaaaccctggacaatgacatcatggaattggatgaggccgcgttctaa

SEQ ID NO: 14 - protein, 617 aa, Mycobacterium avium
MHHHHHHSHYKSNVRDQEFNLFEVLGVDKALGQGEFSDLDADTAREMLNEISRLAEGPIADSFVEGDRNPPVFDPKTHSVTLPESFKKSVHAVIEAGWDKVGIDEALGGVAMPKALLWALHEHILGANPAVWMYAGGAGFANILYHLGTDEQKKWAVMAAERGWGSTMVLTEPDAGSDVGAGRTKAVKQDDGSWHIDGVKRFITSADSDDLFENIFHLVLARPEGAGPGTKGLSLFFVPKFLFDFETGELGERNGVFVTNVEHKMGLKVSATCELSFGQHDVPAKGWLVGEVHNGIAQMFEVIEQARMMVGTKAIATLSTGYLNALEYAKSRVQGADMTQMTDKTAPRVTITHHPDVRRSLMTQKAYAEGLRALYLFTSTYQDAAVAEALYGVDAELAVKVNDLMLPVVKGVGSEQAYAKLTESLQTFGGSGFLQDYPIEQYIRDSKIDSLYEGTTAIQAQDFFFRKIVRDKGVALAHVSEQIQKFVDSESGNGRLKSERELLAKALADVQGMATALTGYLMAAQEDVTSLYKVGLGSVRFLMSVGDLIIGWLLQRQAAVAVAALDAGASGEDRSFYEGKIAVASFFAKNFLPMLTSTREVIETLDNDIMELDEAAF

SEQ ID NO: 15 - DNA, 20 bp, Mycobacterium avium
  primer_bind (1)..(20): qPCR IS900 forward primer
ggcaagaccgacgccaaaga

SEQ ID NO: 16 - DNA, 20 bp, Mycobacterium avium
  primer_bind (1)..(20): qPCR IS900 reverse primer
gggtccgatcagccaccaga

SEQ ID NO: 17 - DNA, 3105 bp, Mycobacterium avium
  CDS (1)..(3099): MAP fusion gene encoding MAP1507-MAP1508- MAP3783-MAP3784-map3694C fusion polypeptide Residues 2-7 His tag; residues 1,8-105 MAP1507; 114-211 MAP1508; 220-316MAP3783; 326-421 MAP3784; 422- 1032; Residues 106-113/212-219/317-325 spacers
atgcatcatcaccatcaccactctttcgtgaccacccaaccagaggccctggctgccgcagcgggcaatctgcaggcgattggctggaccctgagcgcgcagaacgcggcggctgcagcgccaaccacgggtgtggtgccggcagcggccgatgaagtgtccgcgctgacggcggcacagtttgcggcctatgcgcagatgtatcaggcagtgagcgcgcaagcggcagcgattcatgaagcgttcgtcaacaccctgagcaccagctctgtttcctatgccgcgaccgaagcagccaatgccgcagctgcgctgtctgcgtgctactgcgagctgtcgatggcgacgcgttttatgaccgatccgcatgaaatgcgcgctatggccggtcgcttcgaggtccacgcccaaaccgttgaagatgaagcccgcaagatgtgggcgagcagcatgaacattgctggtgccggctggtcaggtcaagcccaagctacgagctacgacaccatgggtcaagttaaccaggcgttccgcaatatcgtgaatatgctgcatggtgttcgtgacggtctgattcgcgacgcaaataactacgaacagcaagagcaagcatctcagcagattctgagcagcggcggttcgggtggcggcagcggtatgtcgatgctggacgcgcacattccgcagctggtcgcggctcagagcgcatttagcgcaaaggctgcgctgatgcgtagcacgatcagccaggcagaacaagaagccgtaagcgcacaagcgttccaccaaggtgagagctctgcggcgttccaagcagcacacgctcgttttgttgaagtcgcagcacgtgtgaacaccctgttggatattgcgcaggcaaacttgggcgatgctgcgggcacctatgttgcggcagacgccgctgcggcgagcggctataccgcgttcggtctggtcccgcgtggtagcaccggtatgagccagattatgtacaattacccggcgatgttgtcccatgctgcagatatgagcggctacgcgggtacgatgcaaggtctgggtgcagacatcgctagcgaacaagccaccctgtccaatgcgtggcagggtgatacgggtatgacctatcaggtgtggcaggcgcagtggaacgaagcgatggagagcctggtgcgtgcgtaccaaagcatggcgtcaacgcacgaagcgaacactatgagcatgttggctcgcgaccaggctgaggcagcgaagtggggtggcatgagccattacaagtctaacgttcgtgatcaagagttcaatttgttcgaggttctgggtgtcgataaggcactgggtcagggtgagttcagcgatctggatgccgacactgcgcgcgagatgcttaacgaaatcagccgtctggcagagggtccaattgccgactcgtttgttgagggtgaccgtaatccgccggttttcgacccgaaaacccattccgtcaccctgccggaaagctttaagaaaagcgttcatgcggttatcgaggcaggctgggataaggttggcatcgacgaagcgctgggcggtgtggcgatgccgaaagcgctgctctgggcgctgcatgagcacatcctgggcgcaaacccggcagtctggatgtatgccggtggtgcgggttttgcgaatatcctgtaccacctgggtaccgacgagcagaaaaagtgggcggttatggcagcggagcgcggctggggttccacgatggtcttgacggagcctgacgcaggcagcgacgtgggtgcgggtcgtacgaaagccgttaagcaagatgacggtagctggcacatcgatggcgtcaaacgtttcatcaccagcgctgactctgatgacctgtttgagaacatcttccacttggttttggcgcgtccggaaggcgcaggccctggcacgaagggtctgagcctgtttttcgtcccgaaattcttattcgatttcgagactggtgagctgggcgagcgtaatggcgtgtttgtcaccaacgtcgagcacaagatgggtctgaaagtgagcgcaacctgtgagctgagcttcggtcaacacgatgttccggcgaaaggctggctggttggtgaggtgcacaacggtatcgcgcagatgtttgaggtgattgaacaggcgcgtatgatggtcggtaccaaggcaattgccaccctgagcacgggttatctgaatgcactggagtacgcgaaaagccgcgtccaaggtgcggacatgacccagatgacggataaaacggctccgcgtgtcaccattacccaccatccggatgtccgtcgcagcctgatgacccaaaaggcttatgccgagggtttgcgtgcgctgtatttgtttacctctacctaccaagacgcggcagtggccgaggccctgtacggtgtggacgccgaattggcggtgaaagttaatgacctgatgctgccggtcgttaaaggtgtgggcagcgaacaggcctacgcgaagttgacggagagcctgcagacctttggcggtagcggttttctgcaggattacccgatcgagcagtatattcgcgatagcaaaatcgatagcctgtacgaaggcacgaccgcgatccaagcccaggactttttctttcgcaagattgttcgtgacaaaggtgtggccctggcgcacgtgagcgagcaaattcaaaagttcgtcgacagcgagagcggcaatggtcgtctgaagtccgagagagagctgctggcgaaagcactggcggatgttcaaggtatggcgactgcgctgacgggttacctgatggctgcacaagaagatgtgaccagcctgtataaagtcggcctgggcagcgttcgtttcctgatgtccgtgggcgatctgatcatcggctggctgctgcagcgtcaggcagctgtcgcggtcgccgcactggacgccggtgcgtccggcgaggaccgcagcttctacgaaggcaagattgccgttgctagcttctttgcgaaaaactttctgccgatgctcacgtccacccgtgaagttatcgaaactctggacaacgacattatggaactggatgaagcggccttttaactcgag

SEQ ID NO: 18 - protein, 1032 aa, Mycobacterium avium
MHHHHHHSFVTTQPEALAAAAGNLQAIGWTLSAQNAAAAAPTTGVVPAAADEVSALTAAQFAAYAQMYQAVSAQAAAIHEAFVNTLSTSSVSYAATEAANAAAALSACYCELSMATRFMTDPHEMRAMAGRFEVHAQTVEDEARKMWASSMNIAGAGWSGQAQATSYDTMGQVNQAFRNIVNMLHGVRDGLIRDANNYEQQEQASQQILSSGGSGGGSGMSMLDAHIPQLVAAQSAFSAKAALMRSTISQAEQEAVSAQAFHQGESSAAFQAAHARFVEVAARVNTLLDIAQANLGDAAGTYVAADAAAASGYTAFGLVPRGSTGMSQIMYNYPAMLSHAADMSGYAGTMQGLGADIASEQATLSNAWQGDTGMTYQVWQAQWNEAMESLVRAYQSMASTHEANTMSMLARDQAEAAKWGGMSHYKSNVRDQEFNLFEVLGVDKALGQGEFSDLDADTAREMLNEISRLAEGPIADSFVEGDRNPPVFDPKTHSVTLPESFKKSVHAVIEAGWDKVGIDEALGGVAMPKALLWALHEHILGANPAVWMYAGGAGFANILYHLGTDEQKKWAVMAAERGWGSTMVLTEPDAGSDVGAGRTKAVKQDDGSWHIDGVKRFITSADSDDLFENIFHLVLARPEGAGPGTKGLSLFFVPKFLFDFETGELGERNGVFVTNVEHKMGLKVSATCELSFGQHDVPAKGWLVGEVHNGIAQMFEVIEQARMMVGTKAIATLSTGYLNALEYAKSRVQGADMTQMTDKTAPRVTITHHPDVRRSLMTQKAYAEGLRALYLFTSTYQDAAVAEALYGVDAELAVKVNDLMLPVVKGVGSEQAYAKLTESLQTFGGSGFLQDYPIEQYIRDSKIDSLYEGTTAIQAQDFFFRKIVRDKGVALAHVSEQIQKFVDSESGNGRLKSERELLAKALADVQGMATALTGYLMAAQEDVTSLYKVGLGSVRFLMSVGDLIIGWLLQRQAAVAVAALDAGASGEDRSFYEGKIAVASFFAKNFLPMLTSTREVIETLDNDIMELDEAAF

(19) United States
(12) Patent Application Publication
(10) Pub. No.: US 2016/0228528 A1
(43) Pub. Date: Aug. 11, 2016

(54) A SINGLE OR MULTISTAGE MYCOBACTERIUM AVIUM SUBSP. PARATUBERCULOSIS SUBUNIT VACCINE

(71) Applicant: JANSSEN SCIENCES IRELAND UC, Co Cork (IE)

(72) Inventors: Gregers JUNGERSEN, Vanløse (DK); Aneesh THAKUR, Frederiksberg (DK); Claus AAGAARD, Copenhagen S (DK); Peter Lawætz ANDERSEN, Brønshøj (DK); Heidi MIKKELSEN, Bagsværd (DK)

(21) Appl. No.: 14/763,742

(22) PCT Filed: Jan. 28, 2014
(86) PCT No.: PCT/EP2014/051645

§ 371 Date: Jul. 27, 2015

(30) Foreign Application Priority Data
Jan. 28, 2013 (EP) 13152908.3

(51) Int. Cl. A61K 39/04; C07K 14/35

(52) U.S. Cl. CPC A61K 39/04; C07K 14/35; A61K 2039/58

(57) ABSTRACT

The present invention provides one or more immunogenic polypeptides for use in a preventive or therapeutic vaccine against latent or active infection in a human or animal caused by a Mycobacterium species, e.g. Mycobacterium avium subsp. paratuberculosis. Furthermore a single or multi-phase vaccine comprising the one or more immunogenic polypeptides is provided for administration for the prevention or treatment of infection with a Mycobacterium species, e.g. Mycobacterium avium subsp. paratuberculosis. Additionally, nucleic acid vaccines, capable of in vivo expression of the multi-phase vaccine comprising the one or more immunogenic polypeptides, is provided for prevention or treatment of infection with a Mycobacterium species, e.g. Mycobacterium avium subsp. paratuberculosis.

### FIELD OF THE INVENTION

The present invention discloses one or more antigenic polypeptides for use in a preventive or therapeutic vaccine against a latent or active infection in a human or animal caused by Mycobacterium spp. such as Mycobacterium avium subsp. paratuberculosis. The invention furthermore discloses a single or multi-phase vaccine comprising the one or more antigenic polypeptides which can be administered either prophylactically or therapeutically for the prevention or treatment of a Mycobacterium spp. infection.

### BACKGROUND OF THE INVENTION

Mycobacteria are capable of causing deadly infections in cattle populations worldwide with major infections caused by Mycobacterium bovis and Mycobacterium avium subsp. paratuberculosis (MAP). MAP is the causative agent of Johne's disease or paratuberculosis, a chronic progressive granulomatous enteric disease infecting young calves either via the oral route or in utero infection (41) and characterized by a long asymptomatic period during which the infection is spread. It may eventually cause wasting, weight loss and death months or years after infection due to severe immune pathology and chronic inflammation in the ileum, ileocaecal valve and associated lymph nodes (4, 40). Consequently, substantial economic losses occur at the farm level due to reduced milk yield, premature culling and reduced slaughter value (26). There is additional growing concern about the presence of MAP in the environment and dairy food products (8) as well as the association of MAP with Crohn's disease in humans (25). Since most animals are exposed/infected in the first days after birth, vaccination prior to infection is often not possible.

There are several findings that show an association of MAP with Crohn's disease (CD) in humans. MAP of bovine origin has been found in CD patients by bacterial culture and genetic probes (42, 43, 44). Profound remissions in CD patients have been induced using anti-mycobacterial drug therapy which has efficacy against MAP (45). MAP reactive T cells have been found in CD patients suggesting a role of mycobacteria in the inflammation seen in CD (46). Multiple studies have identified mutations in the NOD2 gene, a protein that recognizes bacterial molecules and stimulates an immune reaction, and link these to the development of CD (increased susceptibility) (47, 48, 49). According to these findings Crohn's disease may be caused by infection with MAP and MAP infection may be contracted through human ingestion of unpasteurized and pasteurized milk and cattle products.

Moreover, on the basis of these studies, a cure for Crohn's disease may be vaccination against MAP, possibly in combination with anti-mycobacterial antibiotics.

Currently available vaccines against paratuberculosis, such as live attenuated or heat-killed MAP, reduce bacterial shedding but fail to prevent transmission or induce sterilizing immunity (14, 29). Moreover, these vaccines result in false-positive reactors on TB skin testing as well as antibody responses in paratuberculosis diagnostic tests. New MAP vaccines, including subunits (15, 17), DNA (34), expression library immunization (12), and mutant MAP strains (6), have been shown to give only partial protection. A number of putative recombinant MAP proteins have been tested in cattle as potential vaccine candidates including heat-shock protein 70 (Hsp70), members of MAP antigen 85 (Ag85) complex, and superoxide dismutase (SOD). Vaccination with MAP Ag85 complex proteins and SOD in MPL adjuvant induced some protection in calves but no significant differences were observed between vaccinated and non-vaccinated groups (15) while the same antigens in DDA adjuvant induced significant reduction in MAP burden following pre-exposure vaccination in a goat model (50). Also, Hsp70/DDA vaccination has been shown to reduce MAP fecal shedding without affecting tissue colonization compared to non-vaccinated animals (32, 33). Since most calves are infected as neo-natals and vaccines against mycobacterial infections are expected to be imperfect, it is a requirement for optimal effect of a vaccine that it has efficacy as a post-exposure vaccine, and that vaccination does not interfere with other control measures. Diagnosing infected animals in a vaccinated population (the DIVA concept) will thus allow for a much more efficient control of paratuberculosis (51). None of the previously reported live, killed, attenuated or subunit vaccines have shown to provide efficacy following post-exposure vaccination of ruminants AND compliance with current antibody-based surveillance for paratuberculosis and skin-testing for TB. Thus, there exists a need for more effective vaccines for prevention and treatment of paratuberculosis infections in humans and animals, in particular humans and ruminants, while at the same time it is essential that these vaccines do not result in false-positive reactors on TB skin testing or interfere with serological surveillance for paratuberculosis.

### SUMMARY OF THE INVENTION

The invention provides a first immunogenic polypeptide or immunogenic peptide fragment thereof for use as a vaccine wherein said polypeptide comprises an amino acid sequence of SEQ ID NO: 2 or 14; or an amino acid sequence having at least 80% amino acid sequence identity to SEQ ID NO: 2 or 14, wherein administration of the polypeptide or the peptide fragment thereof provides protective immunity in a human or an animal.

In a further embodiment, said first immunogenic polypeptide or immunogenic peptide fragment thereof for use as a vaccine, is combined with at least one additional immunogenic polypeptide or immunogenic peptide fragment thereof, wherein each additional polypeptide has a distinct amino acid sequence selected from the group consisting of SEQ ID NOs: 4, 6, 8 and 10 or immunogenic fragments thereof; or an amino acid sequence having at least 80% amino acid sequence identity to one of SEQ ID NOs: 4, 6, 8 and 10 or the immunogenic fragments thereof. Additionally, at least two of said polypeptides may be comprised within a fusion polypeptide. Administration of the first polypeptide, or peptide fragment thereof, in combination with the at least one additional immunogenic polypeptide, or immunogenic peptide fragment thereof, provides further enhanced protective immunity in a human or an animal, either when administered as individual polypeptides or a fusion polypeptide comprising one or more of said individual polypeptides.

The invention also provides a vaccine for prevention or treatment of a Mycobacterium avium subsp. paratuberculosis infection, wherein said vaccine comprises:
a. The first immunogenic polypeptide; or
b. The first immunogenic polypeptide of (a) combined with at least one additional immunogenic polypeptide, wherein said additional polypeptide has a distinct amino acid sequence having 80% -100% amino acid sequence identity to a sequence selected from the group consisting of SEQ ID NOs: 4, 6, 8, and 10; or
c. the immunogenic polypeptide of (b), wherein at least two of said polypeptides is comprised within a fusion polypeptide; or
d. one or more immunogenic peptide fragment of the immunogenic polypeptide(s) of (a or (b); or
e. one or more nucleic acid molecule encoding the immunogenic polypeptide(s) of (a), (b) or (c), or immunogenic peptide fragment(s) thereof according to (d).
   wherein administration of said vaccine provides protective immunity in a human or an animal.

In a further embodiment, the vaccine comprising two polypeptides having 80% to 100% amino acid sequence identity to a SEQ ID No: 12 and 14, respectively. Alternatively, the vaccine comprising a single fusion polypeptide having 80% to 100% amino acid sequence identity to a SEQ ID No: 18.

The vaccine is suitable for prevention or treatment of a Mycobacterial infection in a human or an animal, in particular an infection selected from among Mycobacterium avium subsp. paratuberculosis M. bovis, M. tuberculosis, M. avium and M. leprae; or alternatively the vaccine is suitable for prevention or treatment of Crohn's disease in a human. The animal may be selected from among a mammal (e.g. porcine, ruminant, equine, feline, canine, primate and rodent), fish, reptile and bird.

The vaccine may further comprise a pharmaceutically acceptable carrier, adjuvant or immunomodulator, which may be selected from among dimethyloctadecylammonium bromide (DDA), dimethyloctadecenylammonium bromide (DODAC), cytosine:phosphate:guanine (CpG) oligodeoxynucleotides, Quil A, polyinosinic acid:polycytidylic acid (poly(I:C)), aluminium hydroxide, oil-in-water emulsions, water-in-oil emulsions (e.g., Montanide, Freund's incomplete adjuvant), IFN-gamma, IL-2, IL-12, monophosphoryl lipid A (MPL), Trehalose Dimycolate (TDM), Trehalose Dibehenate (TDB), monomycolyl glycerol (MMG) and muramyl dipeptide (MDP), mycobacterial lipid extract, nanoparticles or ISCOMs. The carrier can be the adjuvant DDA+TDB.

Where the vaccine comprises one or more nucleic acid molecule encoding the immunogenic polypeptide(s) of (a), (b) (c) or (d), or immunogenic peptide fragment(s) thereof it may be for administration to a mammal by saline or buffered saline injection of naked DNA or RNA or injection of DNA plasmid or linear gene expressing DNA fragments coupled to particles, inoculated by gene gun or delivered by a viral or bacterial vector.

Furthermore, the one or more nucleic acid molecule may be incorporated in the genome of a self-replicating non-pathogenic recombinant carrier, and wherein said carrier is capable of in vivo expression of said immunogenic polypeptides encoded by said more or more nucleic acid molecule. The carrier may be a bacterial or virus carrier.

In one embodiment, the vaccine comprises one or more nucleic acid molecule comprising a nucleic acid sequence selected from among SEQ ID NOs: 1, 3, 5, 7, 9 and 11.

The vaccine of the invention may be for administration to a mammal by parenteral injection.

The invention also provides a method of preparing the vaccine of claims 4 to 11, comprising the step of synthesizing the immunogenic polypeptide of (a), or the immunogenic polypeptide of (a) combined with at least one additional immunogenic polypeptide of (b), or an immunogenic peptide fragment of the immunogenic polypeptide(s) of (a) or (b); solubilizing or dispersing the polypeptide(s) or peptide fragment(s) thereof in an aqueous medium, and optionally adding a pharmaceutically acceptable adjuvant.

### BRIEF DESCRIPTION OF THE DRAWINGS

FIG. 1. A schematic time schedule for comparing the efficacy of vaccination with multi-stage polyprotein (FET11) vaccine and a commercial vaccine Silirum®. Twenty-eight calves, with a mean age of 14 days, were subjected to three doses of experimental MAP inoculations in the first week (starting at 2 weeks of age). The calves were then randomly assigned to four post-infection, vaccination groups comprising early FET11, late FET11, Silirum® and vaccine control groups, as indicated. Calves in early and late FET11 groups were vaccinated with FET11 vaccine twice at the age of 3 and 7 weeks and 16 and 20 weeks, respectively. Silirum® group animals were vaccinated with Silirum® vaccine, once, at the age of 16 weeks. Blood samples were collected at the indicated time points from each calf in the 4 vaccination groups and then evaluated for cell-mediated and humoral immune responses, and the calves were followed up to 52 weeks of age.

FIG. 2. Antigen-specific IFN-γ responses in whole-blood cultures. Levels of IFN-γ released from whole-blood cultures for antigen MAP1507 (a), MAP1508 (b), MAP3783 (c), MAP3784 (d), MAP3694c (e), and CFP10 (f) following vaccination at 3, 7, 16 and 20 weeks as indicated by vertical dotted lines. IFN-γ levels are expressed as mean values (±standard deviation [SD]) for plasma concentrations (μg/ml). Heparinized whole-blood samples were taken from the members of the four vaccination groups, at the time points shown in FIG. 1. The heparin stabilized whole-blood, in 0.5 ml volumes, were stimulated in 48-well culture plates (Greiner Bio-One, Heidelberg, Germany) with each of the five MAP vaccine antigens produced individually in E. coli, or PBS (50 μl volume). In addition, a non-MAP but TB-specific protein, CFP10 (NCBI ref: NP_218391.1) was used at a final concentration of 1 μg/ml in the assay. Whole-blood cultures were incubated for 18-20 h at 37° C. and 5% CO₂ in air. Following overnight incubation, 55p1 heparin solution (10 IU/ml in blood) was added to avoid clots in the supernatant after freezing. Plates were then centrifuged and approximately 0.35 ml of supernatant was collected into 96-well 1-ml polypropylene storage plates (Greiner Bio-One, Heidelberg, Germany) and frozen at −20° C. until analysis. IFN-γ secretion in supernatants was determined by use of a monoclonal sandwich ELISA as described earlier (22). The levels of IFN-γ (μg/ml) were calculated using linear regression on log-log transformed readings from the two-fold dilution series of a reference SEB stimulated plasma standard with predetermined IFN-γ concentration. The IFN-γ responses in PBS cultures were subtracted from MAP vaccine antigen cultures to generate antigen-specific responses.

FIG. 3. Relative quantities of MAP in combined gut tissues as quantified by IS900 qPCR. Standard curve of IS900 qPCR in spiked tissue showing relative quantities (log₁₀RQ) and log₁₀MAP CFU (a), relative quantities of MAP (log₁₀RQ) or IS900 qPCR product for combined ileal tissues (b), and combined jejunum tissues (c). Relative quantities (log₁₀RQ) are expressed as mean values (±standard deviation [SD]) for relative number of MAP (b and c). Group mean value is indicated by solid horizontal line. Data obtained from qPCR (Rotor Gene) was first analyzed through GenEx (MultiD, Goteborg, Sweden) in order to obtain the relative quantities. Based on the dynamic range, a Cq cut-off value of 34 was selected. A Cq value of 34 was assigned to samples with higher Cq or for negative samples. Measured Cq values were then corrected for PCR efficiency to account for suboptimal amplification. Cq values were then converted to a linear scale. As the relative quantity values generated from qPCR analysis were not normally distributed, data were converted to base 10 for statistical analysis with parametric methods. Log-transformed data was compared between the four groups for each of the six selected tissue sites from 4 sites in ileum and 2 sites in jejunum. A comparison was also made between the groups on combined ileal and jejunum tissues. At the same time, relative CFU's of MAP were calculated in the tissues from all the animals based on the standard curve analysis through Rotor Gene software. Protective efficacies of the vaccines were compared by one-way ANOVA followed by Dunn's multiple comparison test using data obtained from both GenEx (log₁₀RQ) or Rotor Gene (CFU).

FIG. 4. PPDj-specific IFN-γ responses in whole-blood cultures. Levels of IFN-γ released from whole-blood cultures stimulated with PPDj throughout the whole study period (a) and between 32-52 weeks of age (b). Whole-blood samples were taken from the members of the four vaccination groups, at the time points shown in FIG. 1, and heparinized. The heparinized whole-blood, in 0.5 ml volumes, were stimulated in 48-well culture plates (Greiner Bio-One, Heidelberg, Germany) with previously added purified protein derivative Johnin (PPDj) (50 μl volume) to a final concentration of 10 μg/ml. Levels of IFN-γ were then measured as described in FIG. 2. IFN-γ levels are expressed as mean values (±standard deviation [SD]) for plasma concentrations (μg/ml).

FIG. 5. Increase in skin thickness of calves of the study following cervical intradermal tuberculin test performed three days prior to slaughter according to European regulations (European Communities Commission regulation 141 number 1226/2002). Results are expressed as mm skin thickness increase for purified protein derivative bovine—Mycobacterium bovis (PPDb) (a), purified protein derivative avian—Mycobacterium avium (PPDa) (b), and mm size difference PPDb-PPDa (c) over a 72 hr period. A threshold of 4 mm over which an animal has a positive reaction, is shown by a horizontal dotted line.

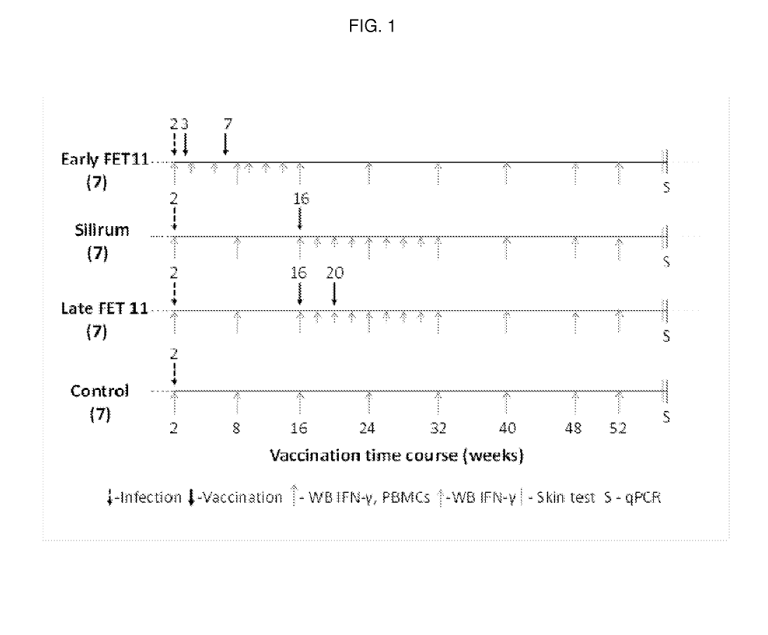

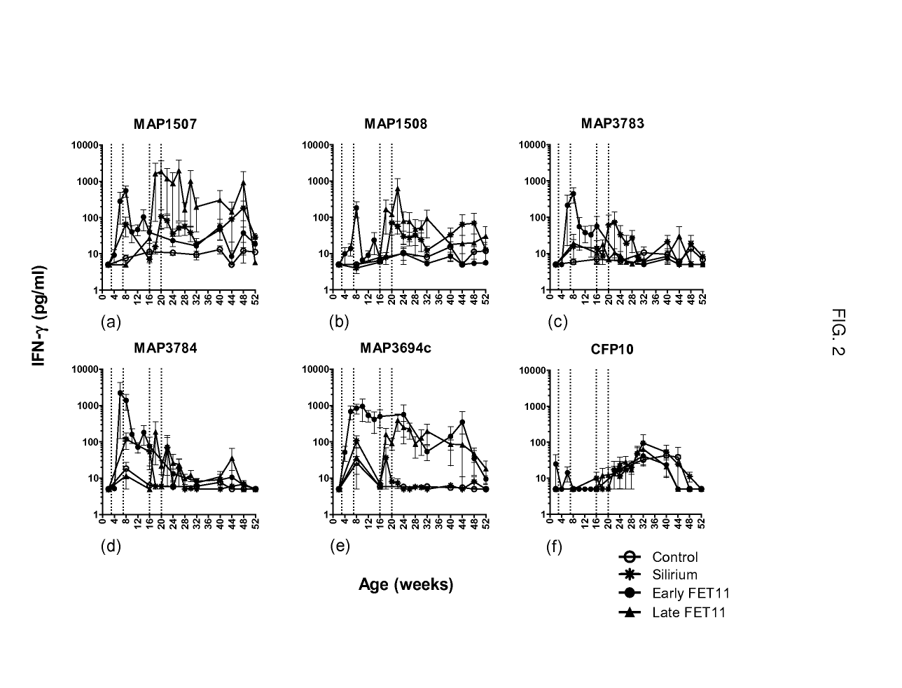

Skin thickness (mm) is given for all individual animals grouped by treatment.

The comparative cervical tuberculin test was conducted as follows: 0.1 ml bovine PPD tuberculin (2000 IU) and 0.1 ml avian PPD tuberculin (2000 IU) (AHVLA; www.defra.gov.uk) were inoculated intradermally in the left side of the neck of each animal. At 72 h post-inoculation, the skin-fold thickness was measured and the increase in the skin-fold thickness compared to day 0 was noted. Reaction to each of the tuberculins was interpreted as follows: a skin test reaction was considered positive when skin thickness increased 4 mm or more, inconclusive when there was an increase of more than 2 mm and less than 4 mm, and negative when the increase was not more than 2 mm.

FIG. 6. Percent seropositive calves with ID Screen® paratuberculosis indirect ELISA (ID Vet, Grabels, France). OD values of serum samples were related to the positive kit control and were interpreted as positive if S/P≧70 percent, as per manufacturer instructions.

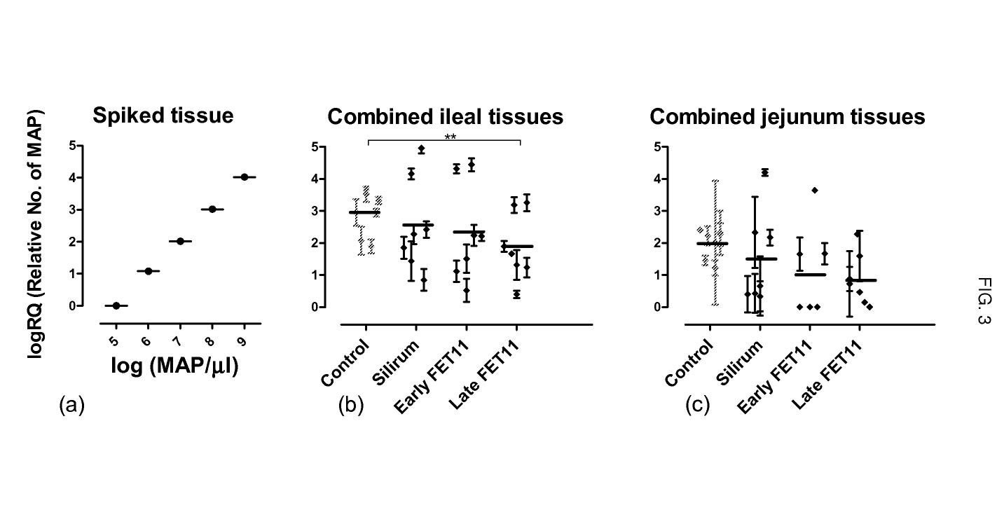

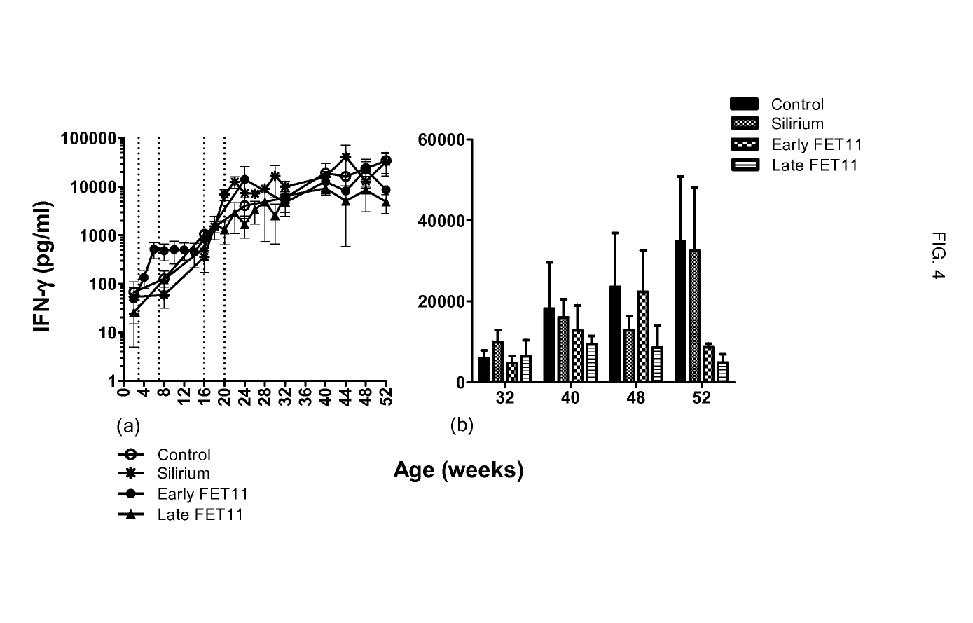

FIG. 7. Antigen-specific IFN-γ responses in whole-blood cultures of MAP inoculated goats at 2 weeks of age (3×10⁹ live MAP bacilli after the three rounds of MAP inoculation). MAP inoculated goats were non-vaccinated controls (controls, n=5) or immunized with 100 μg FadE5 polypeptide (FadE5, n=4), 100 μg FET11 vaccine (FET11, n=5), 100 μg FET13 polypeptide (FET13, n=5) all in CAF09 adjuvant at 14 and 18 weeks post MAP inoculation, as indicated by vertical dotted lines. IFN-γ levels are expressed as mean values (±standard deviation [SD]) for plasma concentrations (pg/ml). Heparinized whole-blood samples were taken from the goats at the time points shown. The heparin stabilized whole-blood, in 0.5 ml volumes, were stimulated in 48-well culture plates (Greiner Bio-One, Heidelberg, Germany) with FET11 polypeptides, FET13 polypeptide or recombinant FadE5 at a final concentration of 1 μg/ml in the assay. Whole-blood cultures were incubated and collected as described in FIG. 2. IFN-γ secretion in supernatants was determined by use of ID Screen® Ruminant IFN-G sandwich ELISA (ID Vet) and IFN-γ production in PBS stimulated cultures was subtracted IFN-γ level in antigen stimulated cultures to provide the antigen-specific response.

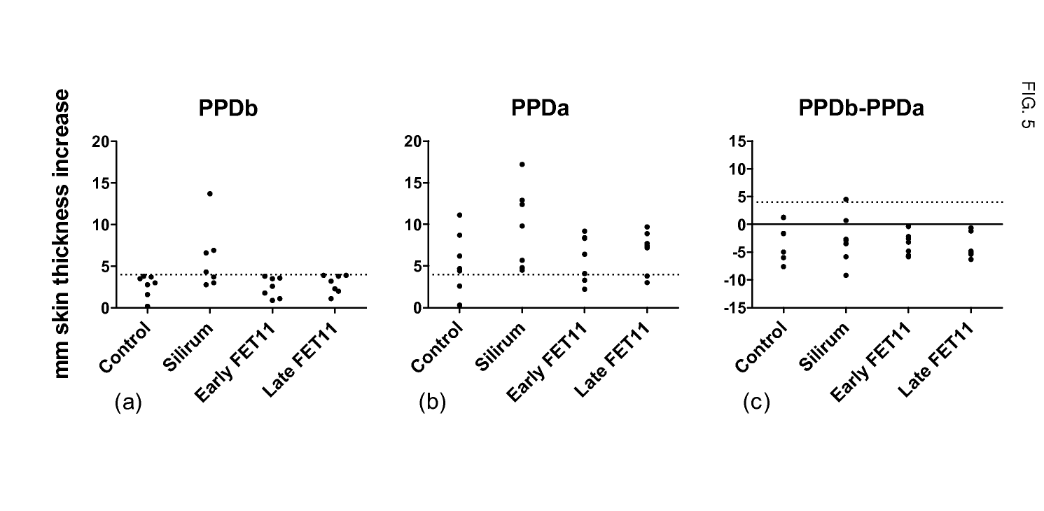

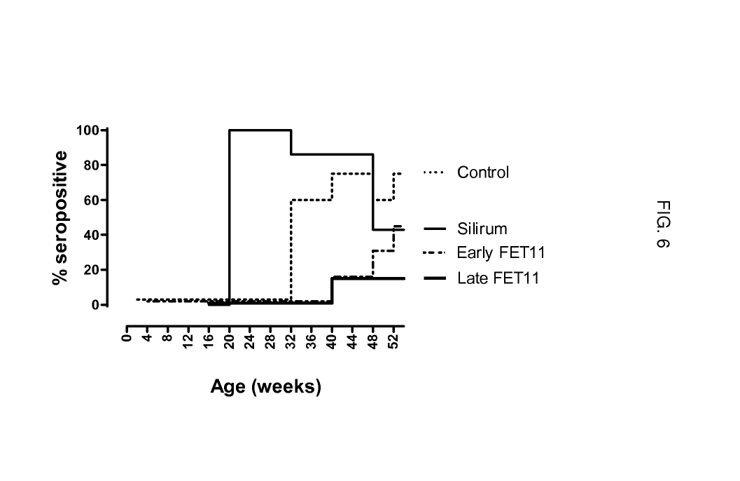

FIG. 8. Relative quantities of MAP in combined gut tissues of goats vaccinated with FET11, FET13 or FadE5 in CAF09 adjuvant and quantified by IS900 qPCR similar to FIG. 3 for calves. Relative quantities of MAP (log₁₀RQ) were combined for multiple samplings at different intestinal locations from each goat with error bars indicating SD of individual values and group mean value indicated by solid horizontal line. The order of goats is identical in all plots. Relative quantities (log₁₀RQ) are expressed as mean values (±standard deviation [SD]) for relative number of MAP at ileocaecal valve (ICV), and 5 samples of ileum/distal jejunum at 0, −25, −50, −75, and −100 cm from ICV for combined ileal tissues (a), jejunum samples at −150, −250, and −350 cm from ICV (b), colon samples at +25 and 50 cm from ICV (c), and ileocoecal lymph node, colonic lymph node, and two samples of mesenterial lymph nodes draining distal half of jejunum (d).

FIG. 9. S/P values in ID Screen® paratuberculosis indirect ELISA (ID Vet, Grabels, France) of calves vaccinated with FET11 polypeptides in CAF01, CAF09 or Montanide ISA61VG adjuvant. OD values of serum samples were related to the positive kit control. Dotted lines indicate positive cut-off at S/P 70 percent and doubtful results at S/P vales between 60 and 70, as per manufacturer instructions.

### DETAILED DESCRIPTION OF THE INVENTION

Although a mycobacterial infection, in particular a M. avium subsp. paratuberculosis infection, commonly occurs at an early age stage of life, the classical clinical case of disease is an adult animal showing no apparent clinical signs, often recognized as the subclinical shedder (37). Thus a mycobacterial infection, e.g. paratuberculosis, has a slow pathogenesis, best described as an initial acute, active phase succeeded by a latent, dormant phase which may persist for long periods of time. During this latent phase, MAP is hidden inside macrophages, undetectable by the immune system.

The present invention lies in selecting one or more immunogenic polypeptide(s), or immunogenic peptide fragment(s) thereof, containing a Mycobacterium avium subsp. paratuberculosis (MAP) antigen that are expressed during specific phases of paratuberculosis infection, and whose use in a vaccine for human and animals is able to stimulate the immune system to generate an immune response leading to a decrease in mycobacterial (e.g. MAP) numbers in tissues and prevent reactivation of a mycobacterial (e.g. MAP) disease, namely a protective immunity. Animals for whom the vaccine provides protective immunity against a mycobacterial infection include mammals (e.g. porcine, ruminant, cattle, equine, feline, canine, primate, and rodent), fish, reptiles and birds. In contrast to existing MAP vaccines used for vaccination, the vaccine of the invention provides surprisingly effective protective immunity against mycobacterial infection (e.g. MAP) in both an uninfected patient (human and animal) as well as in infected patients (human and animal), and at the same time does not interfere with skin-test screening (e.g. TB skin testing) or serological surveillance for a mycobacterial (e.g. MAP) infection. The invention further lies in providing said polypeptides or peptide fragments thereof for use in a vaccine for the treatment of Crohn's disease in a human.

A MAP3694c based vaccine for prevention and treatment of mycobacterial (e.g. MAP) infections in humans and animals.

Accordingly, the present invention provides an immunogenic polypeptide, or an immunogenic peptide fragment thereof which can provide protective immunity, for use as a preventive or therapeutic vaccine, where the polypeptide comprises an amino acid sequence having SEQ ID NO: 2 (or 14) or an immunogenic peptide fragment thereof; or an amino acid sequence having at least 80% amino acid sequence identity to SEQ ID NO: 2 (or 14) or the immunogenic peptide fragment thereof. The polypeptide is known as FadE5 or MAP3694c protein, and is a member of the latency proteins (LATPs) that are expressed by MAP during the latent stage of infection. FadE5 is upregulated in MAP during infection compared to growth in culture, which indicates MAP uses cholesterol as a carbon source during infection as a metabolic adaptation of MAP to the gut environment during infection (52).

Animals infected with MAP that have not received a MAP3694c vaccine, show almost no immune response to MAP3694c, indicating that the MAP3694c is hidden from the immune system during natural MAP infection (see unvaccinated, control calves in Example 3 and FIG. 2, and control goats in FIG. 7). When MAP3694c is administered in a vaccine to a mammal, such as cattle or goats, it is shown to be immunogenic, and induces a significantly elevated IFN-γ response in a vaccinated animal (see FadES vaccinated goats in Example 4 and FIG. 7; and Early FET11 and Late FET11 vaccinated calves in Example 3 and FIG. 2). Furthermore, animals receiving a vaccine comprising MAP3694c show a significant reduction in MAP load in those tissues known to be most prone to harbor MAP in an infection (see FadES vaccinated goats in Example 4 and FIG. 8; and Early FET11 and Late FET11 vaccinated calves compared to unvaccinated, control calves in Example 3 and FIG. 3).

Surprisingly, vaccination with MAP3694c induces an effective immune response even when delivered well after MAP infection is established, and this immune response remains effective at least 8 to 12 months post vaccination. MAP3694c containing vaccines are thus useful for post-exposure vaccine treatment of cows already suffering from clinical paratuberculosis, and that would otherwise have an incompetent immunological response to MAP infection, at least partly due to their inability to detect key antigenic LATP proteins first exposed during disease reactivation.

A single or multi-stage subunit vaccine for prevention and treatment of mycobacterial infection (e.g. MAP) in humans and animals.

In a further embodiment, the invention provides the MAP3694c immunogenic polypeptide, or an immunogenic peptide fragment thereof which provides protective immunity, combined with one to four additional immunogenic polypeptides, where the combination further enhances the protective immunity conferred by the vaccine. Each additional polypeptide has an amino acid sequence selected from the group consisting of SEQ ID NOs: 4, 6, 8 and 10, or immunogenic peptide fragments thereof which provide protective immunity, or alternatively has an amino acid sequence having at least 80% amino acid sequence identity to a sequence selected from among SEQ ID NOs: 4, 6, 8 and 10, or the immunogenic peptide fragments thereof. The immunogenic polypeptide having SEQ ID No: 4, is known as MAP1507 protein. The immunogenic polypeptide having SEQ ID No: 6, is known as MAP1508 protein. The immunogenic polypeptide having SEQ ID No: 8, is known as MAP3783 protein. The immunogenic polypeptide having SEQ ID No: 10, is known as MAP3784 protein. MAP1507, MAP1508, MAP3783 and MAP3784 are all members of the early secretory antigenic target (ESAT) proteins that are expressed by MAP during an early stage of infection or re-infection.

An immunogenic polypeptide according to the invention is either MAP3694c alone, or MAP3694c combined with one or more of MAP1507, MAP1508, MAP3783 and MAP3784, where each polypeptide comprises an amino acid sequence having a preferred minimum percentage of amino acid sequence identity to SEQ ID NO.: 2, 4, 6, 8 and 10 respectively.

The preferred percentage of amino acid sequence identity is at least 80%, such as at least 85%, at least 90%, at least 91%, at least 92%, at least 93%, at least 94%, at least 95%, at least 96%, at least 97%, at least 98%, at least 99%, and at least 99.5%. Preferably, the numbers of substitutions, insertions, additions or deletions of one or more amino acid residues in the polypeptide is limited, i.e. no more than 1, 2, 3, 4, 5, 6, 7, 8, 9, or 10 substitutions, no more than 1, 2, 3, 4, 5, 6, 7, 8, 9, or 10 insertions, no more than 1, 2, 3, 4, 5, 6, 7, 8, 9, or 10 additions, and no more than 1, 2, 3, 4, 5, 6, 7, 8, 9, or 10 deletions compared to the corresponding immunogenic polypeptides having one of SEQ ID NO.: 2, 4, 6, 8 and 10. The term “sequence identity” as used herein, indicates a quantitative measure of the degree of homology between two amino acid sequences of substantially equal length or between two nucleic acid sequences of substantially equal length. The two sequences to be compared must be aligned to best possible fit with the insertion of gaps or alternatively, truncation at the ends of the protein sequences. The sequence identity can be calculated as ((Nref-Ndif)100)/(Nref), wherein Ndif is the total number of non-identical residues in the two sequences when aligned and wherein Nref is the number of residues in one of the sequences. Hence, the DNA sequence AGTCAGTC will have a sequence identity of 75% with the sequence AATCAATC (Ndif=2 and Nref=8). A gap is counted as non-identity of the specific residue(s), i.e. the DNA sequence AGTGTC will have a sequence identity of 75% with the DNA sequence AGTCAGTC (Ndif=2 and Nref=8). Sequence identity can alternatively be calculated by the BLAST program e.g. the BLASTP program (Pearson W. R and D. J. Lipman (1988)) (www.ncbi.nlm.nih.gov/cgi-bin/BLAST). In one embodiment of the invention, alignment is performed with the sequence alignment method ClustalW with default parameters as described by Thompson J., et al 1994, available at http://www2.ebi.ac.uk/clustalw/.

An immunogenic peptide fragment of any one of immunogenic polypeptides MAP3694c, MAP1507, MAP1508, MAP3783 and MAP3784 is a fragment that is capable of inducing an immunogenic response against a Mycobacterium avium subspecies paratuberculosis protein when used as a vaccine according to the invention, and thereby provide protective immunity. When a polypeptide is used for vaccination purposes, it is not necessary to use the whole polypeptide, since an immunogenic peptide fragment of that polypeptide is capable, as such or when formulated with additional immunogenic polypeptides of the invention, of inducing an immune response against that MAP protein and protective immunity.

A variety of techniques are currently available to easily identify immunogenic fragments (antigenic determinants). The method described by Geysen et al (Patent Application WO 84/03564, Patent Application WO 86/06487, U.S. Pat. No. 4,833,092, Proc. Natl Acad. Sci. 81: 3998-4002 (1984), J. Imm. Meth. 102, 259-274 (1987), the so-called PEPSCAN method is an easy to perform, quick and well- established method for the detection of epitopes; the immunologically important regions of the protein. This (empirical) method is especially suitable for the detection of B-cell and T-cell epitopes. Also, given the sequence of the gene encoding any protein, computer algorithms are able to designate specific protein fragments as the immunologically important epitopes on the basis of their sequential and/or structural agreement with known epitopes. The determination of these regions is based on a combination of the hydrophilicity criteria according to Hopp and Woods (Proc. Natl. Acad. Sci. 78: 38248-3828 (1981)), and the secondary structure aspects according to Chou and Fasman (Advances in Enzymology 47: 45-148 (1987) and U.S. Pat. No. 4,554,101). T-cell epitopes can likewise be predicted from the sequence by computer with the aid of Berzofsky's amphiphilicity criterion (Science 235: 1059-1062 (1987) and U.S. patent application Ser. No. 07/005,885). A condensed overview is found in: Shan Lu on common principles: Tibtech 9: 238-242 (1991), Good et al on Malaria epitopes; Science 235: 1059-1062 (1987), Lu for a review; Vaccine 10: 3-7 (1992), Berzofsky for HIV-epitopes; The FASEB Journal 5: 2412-2418 (1991).

In order to directly identify relevant T-cell epitopes, recognized during an immune response, it is possible to use overlapping oligopeptides for the detection of MHC class II epitopes, preferably synthetic, having a length of e.g. 20 amino acid residues derived from the polypeptide. These peptides can be tested in biological assays (e.g. the IFN-γ assay as described herein) and some of these will give a positive response (and thereby be immunogenic) as evidence for the presence of a T cell epitope in the peptide. For the detection of MHC class I epitopes, it is possible to predict peptides that will bind (Stryhn et al. 1996 Eur. J. Immunol. 26 1911-1918.) and hereafter produce these peptides synthetically and test them in relevant biological assays e.g. the IFN-γ assay as described herein. An immunogenic fragment usually has a minimal length of 6, more commonly 7-8 amino acids, preferably more then 8, such as 9, 10, 12, 15 or even 20 or more amino acids.

The combination of MAP3694c immunogenic polypeptide, or immunogenic peptide fragments thereof, and one to four additional immunogenic polypeptides selected from among MAP1507, MAP1508, MAP3783 and MAP3784, or immunogenic peptide fragments thereof, provides a multi-stage subunit vaccine, since the corresponding MAP proteins are expressed during at least two different phases of MAP infection. This multi-stage subunit vaccine provides superior long-term preventive and therapeutic treatment for both newly infected and chronic naturally infected cattle.

The therapeutic effect of vaccination with a vaccine comprising both the MAP3694c polypeptide and a polypeptide comprising the amino acid sequences of all of MAP1507, MAP1508, MAP3783 and MAP3784 polypeptides (as exemplified by FET11 vaccine) is demonstrated herein by treatment of calves following experimental infection with MAP (see example 3). Calves post-exposure vaccinated with the multi-stage subunit vaccine at 4 months of age showed a mean 1.1 log₁₀ reduction in MAP colonization of the gut in comparison with non-vaccinated animals at 8-12 months post MAP infection. Further, compared to calves vaccinated at 3 weeks of age, these older animals developed a more robust immune response and were better able to control MAP load in the tissues even when the infection occurred at an earlier age. Notably, animals vaccinated with FET11 vaccine at an older age were much better able to limit MAP infection in gut tissues as compared to a commercial whole-cell heat inactivated vaccine or early FET11 vaccinated animals.

A discrete immunological profile for the five constituent vaccine polypeptide sequences present in FET11, in both early and late vaccinated animals was observed (Example 3.3). In particular, MAP 1507, MAP1508 and a latency protein, MAP3694c were found to be highly immunogenic. Significant differences in the vaccine-induced Cell-Mediated Immune CMI responses to component vaccine proteins were observed between late FET11 vaccine group and control group over a long period after experimental challenge. These vaccine-induced IFN-γ responses were antigen-specific, as indicated by the fact that control animals did not show any antigen-specific immune response following challenge. In the late FET11 vaccine group, immunogenicity and longevity of vaccine-induced immune responses were clearly evident for MAP1508, MAP1507, and MAP3694c. In vaccinated calves, there was also a strong correlation between total IFN-γ production in response to the pool of MAP3694c, MAP1507, MAP1508, MAP3783 and MAP3784 polypeptides present in the FET11 vaccine and the reduction in bacterial burden in the gut tissues after vaccination. This is consistent with existing evidence that Th1 type immune responses, chiefly IFN-γ , are essential for immunity against mycobacterial infections including MAP (9, 36). Strikingly, calves vaccinated with a commercial whole-cell heat inactivated vaccine and un-vaccinated calves failed to develop any (CMI) response to the polypeptides MAP3784 and MAP3694c, as judged by measurable IFN-γ response. Accordingly, the polypeptides incorporated into the multi-stage subunit vaccine clearly play a critical role in the improved efficacy of this vaccine, particularly when compared to a commercial whole-cell heat inactivated vaccine.

The single or multi-stage subunit vaccine does not interfere with TB testing A known problem of vaccination against paratuberculosis using existing vaccines is that they interfere with diagnosis of tuberculosis on skin testing. Importantly, the single or multi-stage subunit vaccine (as exemplified by FET11 vaccine) did not cause a significant induction of false positive reactors in the intradermal tuberculin test for bovine tuberculosis (see example 3.6), and therefore clearly superior to a commercial whole-cell heat inactivated vaccine that gave false positive reactions. Thus, an inference can be drawn that this vaccine will not interfere with the official diagnostic test for tuberculosis when comparative skin test is used. Adjuvants suitable for use in the single or multi-stage subunit vaccine of the invention, include CAF01, CAF09 and Montanide since FET11 vaccination in these adjuvants does not interfere with TB testing (Example 5).

The single or multi-stage subunit vaccine does not interfere with antibody-based mycobacterial e.g. paratuberculosis testing

A known problem of vaccination against mycobacterial infections, e.g. paratuberculosis, using existing vaccines is that they interfere with antibody-based tests for paratuberculosis, which invalidates their use during a test-and-cull supported eradication program. Importantly, the single or multi-stage subunit vaccine (as exemplified by FET11 vaccine) did not cause any seroconversion in the ID Screen® paratuberculosis indirect ELISA which is used in the Danish “Operation paratuberculosis” eradication program (see example 3.7).

Therefore the multi-stage vaccine is clearly superior to a commercial whole-cell heat inactivated vaccine that induced 100% seropositive animals after vaccination. Thus, an inference can be drawn that this vaccine will not interfere with the antibody-based surveillance program which allows for an improved vaccine-supported eradication campaign as described by Lu et al. (51). Similarly, a number of adjuvants, including CAF01, CAF09 and Montanide, can be used in the single or multi-stage subunit vaccine, without interfering with antibody-based tests for paratuberculosis (see example 5).

Fusion Polypeptides Comprising the Subunits of the Multi-Stage Subunit Vaccine

According to a further embodiment, the subunit vaccine comprises MAP3694c immunogenic polypeptide, or an immunogenic fragment thereof, and at least one additional polypeptide selected from among MAP1507, MAP1508, MAP3783 and MAP3784 (or immunogenic peptide fragments thereof), wherein at least two of said polypeptides is comprised within a single fusion polypeptide. For example, the fusion polypeptide can advantageously comprise the MAP1508 and MAP1507 polypeptide (or immunogenic peptide fragments thereof), and optionally MAP3694c as well. Alternatively, the vaccine may comprise MAP3694c immunogenic polypeptide (SEQ ID No: 2 or 14) (or immunogenic peptide fragments thereof) as a first polypeptide combined with a fusion polypeptide having SEQ ID No: 12 comprising the amino acid sequences of MAP1507, MAP1508, MAP3783 and MAP3784 (or immunogenic peptide fragments thereof), as exemplified by FET11 (see example 1). As another example the fusion polypeptide can comprise MAP3694c, MAP1507, MAP1508, MAP3783 and MAP3784 polypeptides in a single construct (FET13) having SEQ ID NO: 18.

In one embodiment the amino acid sequence of the fusion polypeptide, comprising two or more subunits of the multi-stage subunit vaccine, has an preferred percentage of amino acid sequence identity of at least 80%, such as at least 85%, at least 90%, at least 91%, at least 92%, at least 93%, at least 94%, at least 95%, at least 96%, at least 97%, at least 98%, at least 99%, or at least 99.5% to SEQ ID NO: 12 or 18.

In the present context, the term “fusion polypeptide” is understood as comprising two or more immunogenic polypeptides (or immunogenic peptide fragments thereof) fused together in random order, with or without an intervening amino acid spacer(s) of arbitrary length and sequence. The inclusion of intervening amino acid spacer sequences between any two component immunogenic polypeptides (or immunogenic fragments) serves to enhance recombinant expression of the fusion proteins, and/or enhances the folding of each component protein into its natural form. The delivery of selected candidates as a fusion protein is believed to have the benefit of enhancing immunogenic responses to each of the component immunogenic polypeptides in the vaccine.

Amino acid spacers within the fusion polypeptides of the multi-stage subunit vaccine Amino acid spacer(s) are preferably flexible amino acid stretches and/or do not affect the intrinsic properties of the component immunogenic polypeptides according to the present invention. Preferably, such amino acid spacer(s) are less than 50, even more preferably less than 45, even more preferably less than 40, even more preferably less than 35, even more preferably less than 30, even more preferably less than 25, even more preferably less than 20, even more preferably less than 15, even more preferably less than 10 amino acids long.

Alternatively or in addition, the amino acid spacer(s) have an amino acid length of 1 amino acid or more, 2 amino acids or more, 3 amino acids or more, 4 amino acids or more, 5 amino acids or more, 6 amino acids or more, 7 amino acids or more, and/or 8 amino acids or more. Amino acid spacer(s) of the present invention may thus have for example an amino acid length in the range of 2 to 50 amino acids, 2 to 30 amino acids, 3 to 25 amino acids, 4 to 16 amino acids, 4 to 12 amino acids or any other combination of amino acids lengths disclosed above for peptide linkers. Particularly preferred are peptide linker lengths of 1 to 8 amino acids, e.g. 4 or 8 amino acids.

In terms of amino acid sequence, the amino acid spacer(s) of the present invention are preferably glycine (G) rich peptide linkers, i.e. are amino acid sequences with a high glycine content of more than 50%; e.g. from at least 60 to 80%, for example of about 75%, as exemplified by GGSGGGSG. Alternative amino acid spacer(s) may be the thrombin-cleavable 9-mer, GLVPRGSTG, or have the amino acid sequence: SACYCELS. The amino acid spacer(s) form a contiguous peptide bonded amino acid sequence with the adjacent component immunogenic polypeptides to which they are linked by peptide bonds. The fusion polypeptide of the FET11 and FET13 vaccine comprises amino acid spacers of the above type.

Preparation of the Single or Multi-Stage Subunit Vaccine for MAP

In general the immunogenic polypeptides (herein including fusion polypeptides) of the invention, and DNA sequences encoding such polypeptides, may be prepared by use of any one of a variety of procedures.

The polypeptide may be produced recombinantly using a DNA sequence encoding the polypeptide, which has been inserted into an expression vector and expressed in an appropriate host. Examples of host cells are E. coli. The polypeptides can also be produced synthetically having fewer than about 100 amino acids, and generally fewer than 50 amino acids and may be generated using techniques well known to those ordinarily skilled in the art, such as commercially available solid-phase techniques where amino acids are sequentially added to a growing amino acid chain.

The polypeptides may also be produced with an additional fusion partner, by which methods superior characteristics of the polypeptide of the invention can be achieved. For instance, fusion partners that facilitate export of the polypeptide when produced recombinantly, fusion partners that facilitate purification of the polypeptide. In order to facilitate expression and/or purification, the fusion partner can e.g. be a bacterial fimbrial protein, e.g. the pilus components pilin and papA; protein A; the ZZ-peptide (ZZ-fusions are marketed by Pharmacia in Sweden); the maltose binding protein; gluthatione S-transferase; (-galactosidase; or poly-histidine). Interesting fusion polypeptides are polypeptides of the invention, which are lipidated so that the immunogenic polypeptide is presented in a suitable manner to the immune system. This effect is e.g. known from vaccines based on the Borrelia burgdorferi OspA polypeptide as described in e.g. WO 96/40718 A or vaccines based on the Pseudomonas aeruginosa OprI lipoprotein (Cote-Sierra 3 1998). Another possibility is N-terminal fusion of a known signal sequence and an N-terminal cysteine to the immunogenic polypeptide. Such a fusion results in lipidation of the immunogenic fusion polypeptide at the N-terminal cysteine, when produced in a suitable production host.

Formulation of the single or multi-stage subunit vaccine for mycobacterial disease e.g. paratuberculosis In a further embodiment the invention provides a prophylactic or therapeutic vaccine for treatment of a mycobacterial disease e.g. paratuberculosis comprising a MAP3694c immunogenic polypeptide (or immunogenic peptide fragments thereof), and at least one additional polypeptide selected from among MAP1507, MAP1508, MAP3783 and MAP3784 (or immunogenic peptide fragments thereof). In order to ensure optimum performance of such a vaccine composition it is preferred that it comprises an immunologically and pharmaceutically acceptable carrier, vehicle or adjuvant.

Suitable carriers are selected from the group consisting of a polymer to which the polypeptide(s) is/are bound by hydrophobic non-covalent interaction, such as a plastic, e.g. polystyrene, or a polymer to which the polypeptide(s) is/are covalently bound, such as a polysaccharide, or a polypeptide, e.g. bovine serum albumin, ovalbumin or keyhole limpet haemocyanin. Suitable vehicles are selected from the group consisting of a diluent and a suspending agent. The adjuvant is preferably selected from the group consisting of among dimethyloctadecylammonium bromide (DDA), dimethyloctadecenylammonium bromide (DODAC), cytosine:phosphate:guanine (CpG) oligodeoxynucleotides, Quil A, polyinosinic acid:polycytidylic acid (poly(I:C)), aluminium hydroxide, oil-in-water emulsions, water-in-oil emulsions (e.g. Montanide, Freund's incomplete adjuvant), IFN-gamma, IL-2, IL-12, monophosphoryl lipid A (MPL), Trehalose Dimycolate (TDM), Trehalose Dibehenate (TDB), monomycolyl glycerol (MMG) and muramyl dipeptide (MDP), mycobacterial lipid extract, nanoparticles or ISCOMs. The carrier can be the adjuvant DDA+TDB.

Other methods of achieving an adjuvant effect for the vaccine include use of agents such as aluminum phosphate, aluminum sulphate (alum), synthetic polymers of sugars (Carbopol), aggregation of the protein in the vaccine by heat treatment, aggregation by reactivating with pepsin treated (Fab) antibodies to albumin, mixture with bacterial or protozoan cells such as C. parvum or endotoxins or lipopolysaccharide components of gram-negative bacteria, emulsion in physiologically acceptable oil vehicles (including w/o and w/o/w emulsions) such as mannide mono-oleate (Aracel A) or emulsion with 20 percent solution of a perfluorocarbon (Fluosol-DA) used as a block substitute may also be employed. Other possibilities involve the use of immune modulating substances such as TDB, TDM, MPL or TLR4 agonists or synthetic IFN-gamma inducers such as poly I:C in combination with the above-mentioned adjuvants.

Preparation of vaccines which contain polypeptides as active ingredients is generally well understood in the art, as exemplified by U.S. Pat. Nos. 4,608,251; 4,601,903; 4,599,231 and 4,599,230, all incorporated herein by reference.

Immunization protocol and dosage for single or multi-stage subunit vaccine for a mycobacterial disease e.g. paratuberculosis

The vaccines are administered in a manner compatible with the dosage formulation, and in such frequency and amount as will be prophylactic or therapeutically effective and immunogenic. The single or multi-stage subunit vaccine for a mycobacterial disease e.g. paratuberculosis can be administered as a pre-infection vaccine, but in infected patients (e.g. cattle) will be given as a post-infection vaccine, where it also provides an excellent protection against re-activation of the mycobacterial (e.g. MAP) infection (see examples 3 and 4). According to one embodiment a standard immunization protocol may include a primary vaccination at the age of 0-12, 2-10, 3-8, or preferably 4-6 months followed by a single booster vaccine administered 1, 2, 3, 4, 5, 6, 7 or 8 weeks later, preferably after 4 weeks. Annual booster vaccinations following up on this basic 2-dose vaccination may very likely also be beneficial for this vaccine. If the animal or human does not receive the basic 2-dose vaccination as a juvenile (e.g. calf), the vaccine regimen can be initiated at any age thereafter.

The quantity to be administered depends on the age and weight of the subject to be treated, including, e.g., the capacity of the individual's immune system to mount an immune response, and the degree of protection desired. Suitable dosage ranges are of the order of several hundred micrograms of the polypeptides of the single or multi-stage subunit vaccine per vaccination with a preferred range from about 0.1 μg to 1000 μg, such as in the range from about 1 μg to 300 μg, and especially in the range from about 4 μg to 100 μg.

Administration of the Single or Multi-Stage Subunit Vaccine for a Mycobacterial Disease e.g. Paratuberculosis

Any of the conventional methods for administration of a vaccine are applicable, including oral, nasal or mucosal administration in either a solid form containing the active ingredients (such as a pill, suppository or capsule) or in a physiologically acceptable dispersion, such as a spray, powder or liquid, or parenterally, by injection, for example, subcutaneously, intradermally or intramuscularly or transdermally applied. Vaccine formulations for oral or nasal delivery, which induce mucosal immunity, are also suitable, such as formulations comprising cholera toxin (CT) or its B subunit, which serves to enhance mucosal immune responses and induces IgA production. Modified toxins from other microbial species, which have reduced toxicity but retained immunostimulatory capacity, such as modified heat-labile toxin from Gram-negative bacteria or staphylococcal enterotoxins may also be used to generate a similar effect, and are thus particularly suited in vaccine formulations for mucosal administration.

Additional formulations which are suitable for other modes of administration include suppositories and, in some cases, oral formulations. For suppositories, traditional binders and carriers may include, for example, polyalkalene glycols or triglycerides; such suppositories may be formed from mixtures containing the active ingredient in the range of 0.5% to 10%, preferably 1-2%. Oral formulations include such normally employed excipients as, for example, pharmaceutical grades of mannitol, lactose, starch, magnesium stearate, sodium saccharine, cellulose, magnesium carbonate, and the like. These compositions take the form of solutions, suspensions, tablets, pills, capsules, sustained release formulations or powders and advantageously contain 10-95% of active ingredient, preferably 25-70%.

The invention also pertains to a method for immunizing a human or an animal against a mycobacterial disease e.g. paratuberculosis, comprising administering to the subject (human or animal) the single or multi-stage subunit vaccine or vaccine composition of the invention as described herein.

The invention also pertains to a method for producing an immunogenic composition according to the invention, the method comprising preparing, synthesizing or isolating a therapeutic vaccine for treatment of a mycobacterial disease e.g. paratuberculosis comprising a MAP3694c immunogenic polypeptide (or immunogenic peptide fragments thereof) alone, or in combination with at least one additional polypeptide selected from among MAP1507, MAP1508, MAP3783 and MAP3784 (or immunogenic peptide fragments thereof) as described herein; solubilizing or dispersing the polypeptide(s) or peptide fragment(s) in a medium for a vaccine, and optionally adding a carrier, vehicle and/or adjuvant substance.

Nucleic acid sequences encoding the immunogenic polypeptides of the single or multi-stage subunit vaccine

According to one embodiment, the invention provides a nucleic acid sequence encoding each of the immunogenic polypeptide MAP3694c, MAP1507, MAP1508, MAP3783 and MAP3784 or a fusion thereof that may be used in the preparation of the DNA/RNA vaccine for in vivo expression of these immunogenic polypeptides.

A nucleic acid molecule having SEQ ID No: 1 or 13 encodes MAP3694c; a nucleic acid molecule having SEQ ID No: 3 encodes MAP1507; a nucleic acid molecule having SEQ ID No: 5 encodes MAP1508; a nucleic acid molecule having SEQ ID No: 7 encodes MAP3783; and a nucleic acid molecule having SEQ ID No: 9 encodes MAP3784; a nucleic acid molecule having SEQ ID No: 11 encodes a fusion protein of the FET11 vaccine having SEQ ID No: 12, comprising MAP1507, MAP1508, MAP3783 and MAP3784 as component protein components separated by amino acid spacers; a nucleic acid molecule having SEQ ID No: 17 encodes a fusion protein of the FET13 vaccine having SEQ ID No: 18, comprising MAP3694c, MAP1507, MAP1508, MAP3783 and MAP3784 as component protein components separated by amino acid spacers.

A DNA/RNA Vaccine for In Vivo Expression of the Single or Multi-Stage Subunit Vaccine in a Human or Animal

The invention also relates to a vaccine comprising a nucleic acid fragment, the vaccine effecting in vivo expression of the immunogenic polypeptides of the vaccine in a human or animal, to which the vaccine has been administered, the amount of expressed polypeptide being effective to confer protection or therapeutic treatment of a mycobacterial disease e.g. paratuberculosis infection in a human or animal.

The immunogenic polypeptide may be expressed by a non-pathogenic microorganism (e.g. Mycobacterium bovis BCG, Salmonella and Pseudomonas) or virus (e.g. Vaccinia virus and Adenovirus, Adeno-associated virus, Alphavirus). One or more copies of a DNA sequence encoding one or more immunogenic polypeptide or peptide fragment, as defined above, is incorporated into the genome of the micro-organism in a manner allowing the micro-organism to express and secrete the fusion polypeptide. Another possibility is to integrate the DNA or RNA encoding the one or more immunogenic polypeptide or peptide fragment in an attenuated virus such as the Vaccinia virus or Adenovirus (Rolph et al 1997). The recombinant Vaccinia virus is able to enter within the cytoplasm or nucleus of the infected host cell and the immunogenic polypeptide(s) or peptide fragment(s) of interest can therefore induce an immune response, which is envisioned to induce protection against mycobacterial disease e.g. paratuberculosis. The two most common types of DNA vaccine administration are saline injection of naked DNA or RNA and gene gun DNA/RNA inoculations (DNA/RNA coated on solid gold beads administrated with helium pressure), or delivered by a viral or bacterial vector. Saline intramuscular injections of DNA preferentially generate a Th1 IgG2a response while gene gun delivery tends to initiate a more Th2 IgG1 response. Intramuscular injected plasmids are at risk of being degraded by extracellular deoxyribonucleases, however, the responses induced are often more long-lived than those induced by the gene gun method. Vaccination by gene gun delivery of DNA, to the epidermis, has proven to be the most effective method of immunization, probably because the skin contains all the necessary cells types, including professional antigen presenting cells (APC), for eliciting both humoral and cytotoxic cellular immune responses (Langerhans and dendritic cells). Complete protection from a lethal dose of influenza virus has been obtained with as little as 1 μg DNA in mice. The standard DNA vaccine vector comprises the gene of interest cloned into a bacterial plasmid engineered for optimal expression in eukaryotic cells. Essential structural features of the vector include; an origin of replication allowing for production in bacteria, a bacterial antibiotic resistance gene allowing for plasmid selection in bacterial culture, a strong constitutive promotor for optimal expression in mammalian cells (promoters derived from cytomegalovirus (CMV) or simian virus provide the highest gene expression), a sequence encoding a polyadenylation signal to stabilise the mRNA transcripts, such as a bovine growth hormone (BHG) or simian virus polyadenylation signal sequence, and a multiple cloning site for insertion of an antigen gene. An intron A sequence improves expression of genes remarkably. Many bacterial DNA vaccine vectors contain unmethylated cytidinephosphate-guanosine (CpG) dinucleotide motifs that may elicit strong innate immune responses in the host. In recent years there have been several approaches to enhance and customise the immune response to DNA vaccine constructs (2nd generation DNA vaccines). For instance dicistronic vectors or multiple gene-expressing plasmids have been used to express two genes simultaneously. Specific promoters have been engineered that restrict gene expression to certain tissues, and cytokine/antigen fusion genes have been constructed to enhance the immune response. Furthermore, genes may be codon optimised for optimal gene expression in the host and naive leader sequences may be substituted with optimised leaders increasing translation efficiency.

The administration of a DNA vaccine can be by saline or buffered saline injection of naked DNA or RNA, or injection of DNA plasmid or linear gene expressing DNA fragments coupled to particles, or inoculated by gene gun or delivered by a bacterial or viral vector such as Adenovirus, Modified Vaccinia virus Ankara (MVA), Vaccinia, Adenoassociated virus (AAV), Alphavirus, BCG etc.

Methods for Monitoring the Efficacy of the Vaccine

Quantitative real time PCR (qPCR) provides a rapid and sensitive method for quantification of a mycobacterial infection e.g. MAP, which overcomes detection problems due to the slow growth, long generation time and tendency of mycobacteria (e.g. MAP) to form aggregates. The IS900 element is an insertion sequence considered to be a MAP-specific gene (10). IS900 qPCR is a highly sensitive and specific method for the detection of MAP due to presence of 15-20 copies of IS900 gene within MAP genome (10). qPCR may also be used for monitoring of mycobacterial (e.g. MAP) load in tissues, disease pathogenesis, and efficacy of vaccines and drugs (7, 30). A qPCR method for MAP detection is provided using new PCT primers having SEQ ID No: 15 and 16, that avoid IS900-like sequences and share a relatively close Tₘ, that ensured a higher PCR efficiency. This qPCR assay displayed optimal reaction conditions as evidenced by high efficiency of standard curve for spiked tissue sample (Table 1). Cq values obtained with qPCR assay were analyzed with GenEx® software. GenEx is intuitive software involving sequential analysis of data such as efficiency correction, calibration, normalization, relative quantification etc. and offers distinct advantages over ΔΔCq method. The relative CFU calculation by standard curve method supplemented the results from GenEx analysis, and emphasizes the usefulness of both the approaches.

### EXAMPLES

### Example 1

Cloning and Expression of Mycobacterium avium subsp. Paratuberculosis Antigens

Cloning procedure: The following DNA molecules encoding single or multiple MAP antigens were cloned and expressed:

A DNA molecule (SEQ ID No: 11) encoding a 4-MAP fusion polypeptide (SEQ ID No: 12), comprising the four MAP polypeptides, MAP1507, MAP1508, MAP3783, and MAP3784; and a DNA molecule (SEQ ID No: 13) a single MAP polypeptide, MAP3694c (SEQ ID No: 14); and a DNA molecule (SEQ ID No: 17) encoding a 5-MAP fusion polypeptide (SEQ ID No: 18), comprising the five MAP polypeptides MAP3694c, MAP1507, MAP1508, MAP3783, and MAP3784. The DNA molecules were made synthetically and codon optimized for expression in Escherichia coli (supplied by DNA 2.0, 1140 O'Brien Drive, Suite A, Menlo Park, Calif. 94025, USA). A nucleotide sequence encoding a 6× histidine tag was included at the 5′-end (encoding N-terminus of the polypeptide) of each DNA molecule to facilitate purification of the expressed polypeptides. Following synthesis, each of the DNA molecules were inserted into an expression vector pJexpress 411 (DNA2.0, US) harboring a T7 promoter, a ribosomal binding site and a T7 terminator to facilitate efficient transcription and translation in Escherichia coli, to give recombinant expression vectors: pJ411-fusion 4-MAP, pJ411-fusion 5-MAP and pJ411-MAP3694c, respectively. CFP10 at M. tuberculosis antigen (NCBI ref: NP_218391.1) was cloned in the same vector.

Protein expression and purification procedure: Both proteins were expressed and purified according to the same protocol. Aliquots of Escherichia coli BL21-AI (Invitrogen) cells were transformed with vectors pJ411-fusion 4-MAP, pJ411-fusion 5-MAP and pJ411-MAP3694c, and expression of the encoded recombinant polypeptides was induced by the addition of 0.2% arabinose to the growth medium when the cell culture density reached OD600˜0.5.

After 3-4 hours further growth at 37° C., the bacteria were harvested and lyzed using Bacterial-protein extraction reagent (B-PER: Pierce; Thermo Fisher Scientific Inc. 3747 N Meridian Rd, Rockford, Ill. 61101, USA). Both recombinantly expressed polypeptides formed inclusion bodies, which were washed three times in 20 mM Tris-HCl pH 8.0, 100 mM NaCl, 1 mM EDTA, 0.1% deoxycholic acid and then dissolved in 8 M urea, 100 mM Na₂PO₄, 10 mMTris-HCl pH 8.0 (buffer A) before being applied to metal affinity columns (Clontech Laboratories: 1290 Terra Bella Ave. Mountain View, Calif. 94043, USA) to selectively bind the N-terminal histidine tagged polypeptides. Bound polypeptides were washed five times with two column volumes of buffer, alternating between 10 mM Tris-HCl pH 8.0, 60% isopropanol and 50 mM NaH₂PO₄ pH 8.0, before being eluted in buffer A supplemented with 200 mM imidazole. All fractions were collected and analyzed by SDS-PAGE using Coomassie staining, and inspected for the purity of fractions enriched for the expressed polypeptides.

Relevant fractions were pooled and dialyzed against 3 M urea, 10 mM Tris-HCl pH 8.5 and applied to anion-exchange columns (Pharmacia) washed with 5 column volumes 10 mM Tris-HCl pH 8.5 and eluted using a linear NaCl gradient, from 0 to 1 M over 40 column volumes. Based on purity, fractions were pooled and dialyzed against 20 mM Glycine pH 9.2. Finally, total protein concentration (NanoOrange™ Protein Quantitation Kit, Life Technologies, Denmark) and purity was determined for the two recombinantly expressed polypeptides. CFP10 was expressed and purified according to the same protocol.

### Example 2

Method for Quantification of Mycobacterium avium subsp. paratuberculosis (MAP) in Animal Tissue

An IS900 quantitative Real Time PCR assay was developed to provide an indirect quantitative measure of the number of MAP cells in a tissue sample derived from an animal. The assay is based on the specific detection/quantification of MAP-derived DNA present in DNA extracts of the tissue sample, using Map specific PCR primers and real time PCR, as detailed below. MAP can be distinguished from other members of the M. avium complex by virtue of having 14-18 copies of IS900 inserted into conserved loci in its genome.

2.1 DNA Extraction from Animal Tissue

Samples of animal tissue were homogenized, and 25 mg aliquots were weighed out into tubes and incubated on a shaker incubator overnight at 37° C. in 400 μl Tissue Lysis Buffer (ATL: Qiagen, Valencia, Calif. 91355, USA). Particular attention was given while measuring the weight of the tissue homogenates in order to use the precise amount of homogenized material each time. The incubated homogenates were then subjected to bead beating at full speed for 1 min with 200 μl of 0.1 mm Zirconia/Silica beads (BioSpec Products Inc. USA) to complete cell lysis, and were then centrifuged for 30 sec at 5000×g. After bead-beating, DNA was extracted from the homogenates using the Qiagen DNeasy Blood and Tissue kit and protocol. Samples of the supernatants (200 μl) were transferred into new tubes to which 20 μl proteinase-K was added, and the tubes were then incubated for 10 min at 56° C. The tubes were then centrifuged through a Qiagen Spin Column and washed according to the kit protocol. In the final step, DNA was eluted with 200 μl elution buffer and stored at −20° C. prior to use.

2.2 qPCR Assay for MAP-derived DNA

Primers selectively binding to MAP-specific sites of the IS900, which were designed using Primer3 (http://frodo.wi.mit.edu/cgi-bin/primer3/primer3www.cgi) (31) have the following sequence:

qPCR IS900 forward: 5′-GGCAAGACCGACGCCAAAGA-3′ [SEQ ID NO: 15];

qPCR IS900 reverse: 5′-GGGTCCGATCAGCCACCAGA-3′ [SEQ ID NO: 16]. qPCR reactions were carried out in 25 μl volumes, containing 2.5 μl DNA template, 12.5 μl of 2× QuantiTect SYBR Green PCR Master Mix (Qiagen, supra), and 0.125 μl of 10 μM of each of the forward and reverse primers in nuclease free water. Quantitative PCR (qPCR) was performed on a Rotor Gene Q PCR system (QIAGEN, supra). PCR cycling started with an initial denaturation at 95° C. for 15 min, followed by 45 cycles of amplification at 95° C. for 30s and 68° C. for 60s. After PCR amplification, melting curve data were collected and analyzed.

2.3 qPCR Assay Performance: Dynamic Range and Specificity for Quantification of MAP in Tissue Samples

A standard curve was made by spiking samples of tissue DNA (prepared from tissue sample taken from jejunum located 250 cm proximal distance from the ileocaecal valve obtained from an animal found to be consistently qPCR MAP-negative) with DNA from MAP culture (1×10⁹ CFU/ml MAP Ejlskov). 2 μl of bacterial culture DNA in serial ten-fold dilutions of 1×10⁹ organisms was used to spike the tissue DNA samples.

Key performance characteristics of qPCR assay are summarized in table 1. The R2 value for the spiked tissue standard curve was above 0.99 and the PCR efficiency was 0.97. The detection limit for the spiked tissue was 1.2×10² MAP/g tissues. Detection limit was defined as the concentration giving a positive quantification cycle (Cq) value in one or more of the triplicate samples of the standard curves. The lower limits of the dynamic range were based on the mean of the triplicate values and define the quantification limits of the qPCR assay. The dynamic range was four log units i.e. 1.2×10⁹-1.2×10⁵ CFU/g tissue (see also FIG. 3a).

TABLE 1
Key characteristics of IS900 qPCR assay in spiked tissue samples

| Factor | Spiked tissue |
|---|---|
| PCR efficiency | 0.97 |
| Coefficient of Determination (R²) | 0.997 |
| Dynamic range* (MAP CFU/g tissue) | 1.2 × 10⁹-1.2 × 10⁵ |
| Detection limit (MAP CFU/g tissue) | 1.2 × 10² |

*Range of linearity of the standard curve
CFU: Colony Forming Units

### Example 3

### Multistage Subunit Vaccine Versus a Commercial Whole Cell MAP Vaccine

This study demonstrates the efficacy of the multistage subunit vaccine of the invention (FET11), compared to a commercially available vaccine, or no vaccination, in providing an efficient immune response that is protective against MAP. The vaccines were tested in MAP infected calves.

3.1 Preparation of the Vaccine FET11 and the Vaccination Protocol

Animals: Male jersey calves were obtained over a period of four months from a dairy farm proven to have a true prevalence equal to, or close to, zero by the Danish paratuberculosis surveillance program (21). A total of 28 calves were acquired with a mean age of 14 days. Animals were housed and raised under appropriate biological containment facilities (BSL-2) located at the institute premises with community pen and straw bedding.

MAP culture: The strain of MAP used for infection of the calves was a Danish clinical isolate, Ejlskov 2007, isolated in 2007 from the faeces of a clinically affected cow. The strain was grown on Lowenstein-Jensen medium (Becton Dickinson, 1 Becton Drive, Franklin Lakes, N.J. 07417, USA) slants and was propagated on Middlebrook 7H9 medium (Becton Dickinson, supra) supplemented with 10% oleic acid-albumin-dextrose complex (Difco; Becton Dickinson supra) plus 0.05% Tween 80 (Sigma-Aldrich Co., 3050 Spruce St. St. Louis, Mo. 63103, USA) and 2% Mycobactin 3 (Allied Monitor Inc., 201 Golden Industrial Drive, Fayette, Mo. 65248 USA) at 37° C. A mid-log-phase culture (OD600 nm) was centrifuged and counted using a pelleted wet weight method that estimates approximately 1×10⁷ colony-forming unit (CFU)/mg pelleted wet weight (11). The cells were validated for purity by performing contamination controls in blood agar plates (37° C., 72 h), Ziehl-Neelsen staining, and IS900 PCR, and subsequently were frozen as 1 ml inoculum aliquots containing 1×10⁹ CFU and 15% glycerin. Two days before inoculation, a 1 ml inoculum aliquot was thawed in a water bath (37° C.), then added to pre-warmed media (20 ml MB7H9 with supplements) and incubated on a shaker at 37° C. for 48 h in order to provide a MAP infection inoculum for each individual calf.

MAP experimental infection: MAP infection was performed by individually feeding calves with a 20 ml MAP infection inoculum in a liter of pre-warmed (38° C.) commercial milk replacer (DLG, Denmark), and repeated MAP infection procedure with a further two times in the first week (i.e. Day 0, 2 and 7) after acquiring each calf (i.e. starting at 2 weeks of age). MAP bacilli in each MAP infection inoculum (48 h, 20 ml culture) were enumerated by serial dilution plate counting on Middlebrook 7H10 agar (Becton Dickinson supra).

Retrospective quantification of CFU's indicated that in total calves received a dose of 2×10¹⁰ live MAP bacilli after the three rounds of MAP infection. All calves used in this study were inoculated with MAP following the above protocol.

Vaccine composition: The multi-stage vaccine, FET11, comprised a recombinantly-expressed fusion polypeptide of four MAP polypeptides (MAP1507, MAP1508, MAP3783, and MAP3784) and a recombinantly-expressed MAP latency-associated polypeptide (MAP3694c) formulated with CAF01 adjuvant. CAF01 is a cationic liposome composed of dimethyldioctadecyl-ammonium bromide (DDA) and trehalose dibehenate (TDB) combined in a wt/wt ratio of 2500 μg/500 μg per dose. The fusion polypeptide and single polypeptide were produced and purified as described in Example 1, and were then allowed to adsorb to the adjuvant CAF01 for 1 h at RT before injection (2).

Silirum® is a commercial MAP vaccine containing 2.5 mg of a heat-inactivated MAP strain 316F culture combined with an adjuvant consisting of highly refined mineral oil (CZ Veterinaria S.A., P.O. Box 16-36400 Porriño, Spain).

Vaccination groups and procedure: The first four calves were randomly assigned to four vaccination groups comprising early FET11, late FET11, Silirum® and vaccine control groups, respectively. Calves were born over a period of 4 months and followed the same grouping sequence according to date of birth. Calves in early and late FET11 groups were vaccinated with FET11 vaccine twice at the age of 3 and 7 weeks and 16 and 20 weeks, respectively. Silirum® group animals received 1 ml of Silirum® vaccine (CZ Veterinaria S.A., P.O. Box 16-36400 Porriño, Spain) at the age of 16 weeks in accordance with the manufacturer instructions. Calves were vaccinated by the sub-cutaneous route in the right mid-neck region about 7 cm ahead of the prescapular lymph node. Calves vaccinated with FET11 received 100 μg fusion polypeptide and 100 μg MAP3694c mixed 1:1 with CAF01 in a total volume of 2 ml. Control calves did not receive any vaccine.

3.2 Clinical Evaluation of Vaccination with FET11 Versus a Commercial Whole Cell MAP Vaccine

No clinical signs indicative of paratuberculosis were observed among the infected calves. No side effects were observed following subcutaneous FET11 vaccination in calves. However, subcutaneous Silirum® vaccination resulted in a transient nodule of approximately 2-2.5 cm, which subsided after about 3 weeks.

3.3 Immunization with FET11 Vaccine Induces Elevated Levels of Antigen-Specific IFN-γ Responses

The immunogenicity and longevity of the FET11 vaccine adjuvanted with CAF01 in the four vaccination groups, was examined by measuring vaccine antigen-specific IFN-γ responses in whole-blood, sampled from calves in each group at multiple time points up to 44 (n=8) or 52 (n=20) weeks of age, as described in FIG. 2. In general, control (non-vaccinated) calves did not show antigen-specific response to the vaccine proteins, with the exception of two calves that responded weakly to MAP3694c and MAP3784, five weeks after MAP infection.

In response to protein MAP1507, IFN-γ levels in early FET11 vaccinated calves peaked at one week after the second vaccination and thereafter remained at low levels throughout the study period. In comparison, late FET11 calves showed immediate increase in levels of IFN-γ after first vaccination that remained consistently higher for a long period but dropped around week 48. Calves vaccinated with Silirum® had consistent MAP1507-specific IFN-γ production after vaccination with levels in between early and late FET11 groups.

In response to MAP1508, IFN-γ levels for early FET11 vaccinated calves declined 3 weeks after second vaccination and remained at low levels afterwards. On the other hand, in both late FET11 and Silirum® vaccine groups IFN-γ levels peaked after vaccination. However, IFN-γ levels that were higher for late FET11 vaccination than Silirum® dropped between weeks 38 up to 48 before coming up again.

In response to MAP3783, early FET11 vaccinated calves had high IFN-γ levels that waned after week 16 while IFN-γ responses for Silirum® calves weakened after week 26. Late FET11 vaccinated calves responded poorly to MAP3783 through all the time points.

Production of IFN-γ against MAP3784 was the highest in early FET vaccinated calves, 7 weeks after the second vaccination, but dropped afterwards. Late FET11 and Silirum® vaccinated calves showed low response to this protein. However, Silirum® vaccinated calves immediately responded to this protein before Silirum® vaccination.

Only calves in early and late FET11 vaccination groups responded to MAP3694c, with similar response in both groups, starting relative to time of vaccination and dropping around week 46. For the non-MAP TB-specific protein CFP10, responses were found to come up after 20 weeks of MAP infection in all vaccine and control groups, though the responses were very low.

MAP vaccine antigen-specific IFN-γ production between different vaccine groups was compared by one-way ANOVA followed by Dunn's multiple comparison test. In terms of levels of significance for FET11 vaccine, the late FET11 vaccine group responded significantly to MAP1507 (p<0.001), MAP1508 (p<0.05) and MAP3694c (p<0.05) as compared to control group. However, early FET11 group only showed a significant response to MAP3694c (p<0.001) in comparison with control group. Both early (p<0.001) and late FET11 (p<0.05) vaccine groups showed a significant difference from the Silirum® group with respect to levels of MAP3694c induced IFN-γ. By contrast, Silirum® vaccine did not show a significant difference in response from the control group to any of the vaccine proteins.

3.4 FET11 Vaccination Lowers Tissue Colonization of MAP

At the end of the 44 weeks (n=8) or 52 weeks (n=20) of age, the calves in the study were euthanized and necropsied. Six tissue samples from each animal were collected and processed for IS900 qPCR: ileocaecal valve, ileum (0 cm, −25 cm, −50 cm; distance indicated relative to the location of ileocaecal valve in proximal direction), and jejunum (−150 cm, −250 cm). The tissue samples (8 cm in length) were rinsed with sterile PBS. Epithelium, submucosa, and lamina propria were scraped from the serosa with a sterile object glass. The tissue scrapings were homogenized by blending in a rotor/stator type tissue homogenizer (Tissue-Tearor from BioSpec Products Inc. 280 North Virginia Avenue, Bartlesville, Okla. 74003 USA). Six tissue samples from the gut of each animal were processed for DNA extraction, and relative quantification of MAP was performed using qPCR (see Example 2). These six tissue sites were selected, as they are more likely to harbor infection based on experimental MAP inoculation studies. Apparently, no significant variation was observed between relative quantities (RQ) of MAP among animals killed at week 44 and week 52. Therefore, data from both time points were combined for analysis as required.

In each of the six selected tissues, a lower mean relative concentration of MAP was observed in the late FET11 vaccinated animals as compared to control group (Table 2).

TABLE 2
Animal-wise relative quantities of MAP in individual gut tissues

| | | | Ileum 0 | Ileum −25 | Ileum −50 | Jejunum −150 | Jejunum −250 |
|---|---|---|---|---|---|---|---|
| Group | Animal | IC Valve | cm | cm | cm | cm | cm |
| MSV-2 Early FET11 | 02204 | 4 | 7 | 5 | 1 | 1 | 1 |
| | 02214 | 520 | 143 | 134 | 89 | 1 | 1 |
| | 02221 | 223 | 148 | 106 | 208 | 81 | 27 |
| | 02234 | 9 | 7 | 40 | 12 | 1 | 1 |
| | 02250 | 15966 | 27464 | 15966 | 27464 | 106 | 19 |
| | 02264 | 25663 | 41251 | 38547 | 14919 | 4114 | 4711 |
| | 02291 | 15 | 13 | 54 | 106 | 1 | 1 |
| MSV-2 Late FET11 | 02201 | 16 | 16 | 102 | 109 | 3 | 3 |
| | 02212 | 958 | 2019 | 3977 | 1438 | 143 | 11 |
| | 02219 | 57 | 38 | 75 | 92 | 14 | 4 |
| | 02229 | 3 | 3 | 2 | 2 | 1 | 1 |
| | 02236 | 59 | 56 | 56 | 64 | 28 | 1 |
| | 02269 | 41 | 21 | 14 | 8 | 1 | 1 |
| | 02299 | 2931 | 1951 | 1214 | 808 | 208 | 170 |
| MSV-2 Silirum | 02197 | 3 | 21 | 7 | 5 | 5 | 1 |
| | 02205 | 64098 | 135131 | 117994 | 68594 | 13476 | 18915 |
| | 02216 | 143 | 48 | 6 | 14 | 7 | 1 |
| | 02225 | 292 | 538 | 239 | 130 | 223 | 99 |
| | 02237 | 78 | 215 | 37 | 42 | 6 | 1 |
| | 02251 | 255 | 439 | 106 | 106 | 21 | 1 |
| | 02279 | 8673 | 18284 | 13027 | 20940 | 1299 | 36 |
| MSV-2 Control | 02198 | 4711 | 4403 | 3357 | 6613 | 2392 | 4 |
| | 02207 | 596 | 958 | 1174 | 958 | 637 | 68 |
| | 02217 | 201 | 371 | 42 | 64 | 37 | 23 |
| | 02227 | 6613 | 3844 | 2739 | 1823 | 273 | 106 |
| | 02238 | 2235 | 1592 | 659 | 273 | 239 | 273 |
| | 02257 | 121 | 66 | 208 | 148 | 19 | 9 |
| | 02281 | 2931 | 2235 | 1951 | 1592 | 335 | 106 |

Relative quantities of MAP IS900 qPCR product for ileocaecal valve, ileum 0 cm, ileum −25 cm, ileum −50 cm, jejunum −150 cm, and jejunum −250 cm (distance in cm indicated relative to the location of ileocaecal valve in proximal direction).
Relative quantities were generated from raw Cq values after correction for PCR efficiency using GenEx software (see also FIG. 3) and then normalized with interplate calibrators based on two identical samples run on all plates.
The two animals at the top of each group were euthanized at 44 weeks of age while all other animals were euthanized at 52 weeks of age.

Silirum® and early FET11 vaccinated animals exhibited comparable relative MAP tissue load (log10RQ) in all six tissues, with two animals from both groups having very high relative MAP numbers. After combining the four ileal tissues and two jejunum tissues, the analysis revealed a similar picture, where late FET11 vaccinated animals showed a mean 1.1 log10 reduction in MAP load as compared to the control group(p<0.01) (FIGS. 3b and 3c). Analysis of CFU generated through standard curve analysis by Rotor Gene also revealed a mean 1.1 log10 reduction in MAP CFU in late FET11 group compared to the control group (p<0.05).

3.5 Johnin Purified Protein Derivative-Specific IFN-γ Production Correlates with MAP Load in Tissues

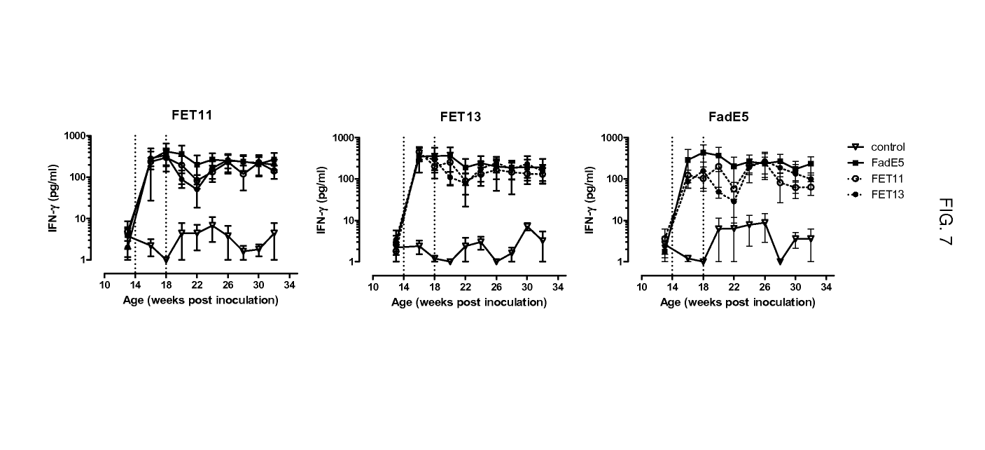

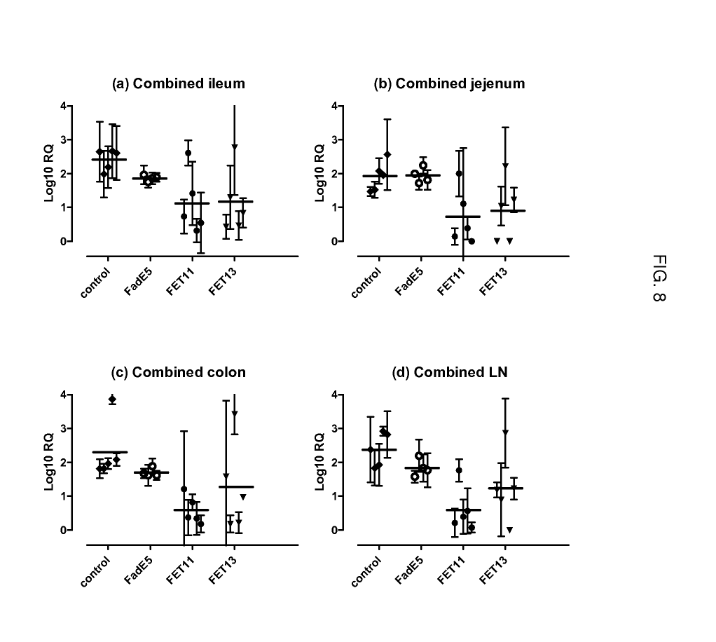

Johnin purified protein derivative (PPDj) is a crude undefined extract of MAP antigens prepared from different MAP strains. All animals exhibited progressive PPDj-specific IFN-γ production following MAP infection (FIG. 4a). Characteristically, in the early FET11 vaccine group these responses declined after the second vaccination but came up again after 8 weeks. At later stages of the study, early FET11 vaccinated animals had lower PPDj-specific responses than Silirum® or control group. Late FET11 vaccinated animals had consistently lower PPDj-specific responses after vaccination. By comparison, the Silirum® and control group animals showed an increasing trend of IFN-γ responses against PPDj towards the late stages of the study (FIG. 4b).

Log-transformed IFN-γ responses to vaccine proteins or PPDj and relative MAP concentration (log₁₀RQ) were correlated by a non-parametric Spearman correlation. Statistical analysis was performed using GraphPad Prism software vs. 5.02 (GraphPad Software Inc., La Jolla, Calif.). P<0.05 was considered statistically significant. A positive linear correlation between the log-transformed PPDj mean IFN-γ responses and relative quantities of MAP (log₁₀RQ) was found at weeks 48 (p<0.05) and 52 (p<0.01) post infection. These results were supplemented by the observation that an inverse statistical correlation was found between the relative quantities of MAP (log₁₀RQ) and log-transformed mean IFN-γ responses against vaccine proteins at weeks 24, 32 and 40 of the study (p<0.05). IFN-γ responses to PPDj also correlated with MAP CFU calculated through a standard curve at weeks 32 (p<0.001), 40 (p<0.05) and 52 (p<0.05) of the study. In terms of vaccine group, there was only a significant correlation between IFN-γ responses to pooled vaccine proteins and MAP load in tissues for late FET11 vaccine group at week 32 (p<0.05). There was a correlation between IFN-γ response to vaccine proteins, MAP1508, MAP1507 and reduced bacterial load at week 22, 24 and 32 (p<0.05). Accordingly, PPDj responses provide a good measure of tracking infection status in experimental MAP infections and thus can serve as a surrogate of infection in vaccinated animals.

3.6 Tuberculin Skin Testing

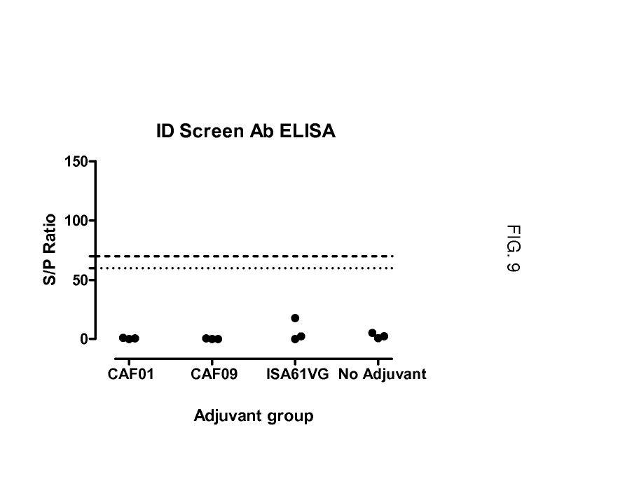

All calves in the study were subjected to comparative intradermal tuberculin testing 72 hrs before slaughter at the end of the week 44 (n=8) or 52 (n=20) (FIG. 5). According to the official guideline, the single intradermal comparative cervical tuberculin test (SICCT) is to be considered: positive, when the positive reaction to bovine PPD is more than 4 mm greater than the reaction at avian site; inconclusive, when the positive reaction to bovine PPD is between 1 to 4 mm greater than the avian reaction; and negative, when there is a negative reaction to bovine PPD or when a positive or inconclusive reaction to bovine PPD is less than or equal to a positive or inconclusive reaction at the avian site.

All of the FET11 vaccinated animals were low PPDb responders and were negative in SICCT. In the Silirum® group, however, four calves had strong positive skin reactions to PPDb, and one animal had a positive SICCT reaction, i.e. it tested (false) positive for bovine tuberculosis.

3.7 Antibody-Based Assays

Serum samples were obtained from all calves in the study and analyzed for reactivity in the ID Screen® paratuberculosis indirect ELISA, which is used in the Danish “Operation paratuberculosis” eradication program (FIG. 6). In this antibody ELISA, sample to positive ratio (S/P values) above 70 are positive, S/P values in the range of 60-70 are doubtful, and S/P values below 60 are negative. No reactivity in the ELISA was observed in response to the Fet11 vaccination with all S/P values from the two Fet11 groups below 13 at any point in the first 30 weeks of the study. In contrast, all Silirum® vaccinated animals produced S/P values from 112-164 at 2 and 6 weeks post vaccination (week 20 and 24). The non-vaccinated control calves responded to the MAP infection with seropositive samples (S/P range 108-188) in 4 of 7 calves at 32 weeks of age and 5 of 7 calves at 40 weeks and onwards. Due to the vaccine-induced seroconversion in Silirum®-vaccinated calves, the antibody ELISA cannot be used to evaluate progression of MAP infection in these animals. In the two groups of Fet11 vaccinated calves, however, only a total of 4 of 14 calves were seropositive at 40 weeks or later in the antibody ELISA, which shows a delayed MAP progression in a reduced number of animals compared to non-vaccinated controls. These results illustrate how antibody-based surveillance for MAP, as currently used in most eradication campaigns, can be continued along with multi-stage vaccination to reduce incidence and progression of MAP. This is not possible with Silirum® or other current vaccines against MAP.

### Example 4

### FadE5 Immunization of Goats

This study demonstrates the immunogenicity and protective effect of different forms of the single-stage and multi-stage MAP vaccine. The immunization was performed in MAP inoculated goats.

4.1 Preparation of the FadE5 Vaccine and the Vaccination Protocol

Animals: One to three weeks old goat kids were obtained from a goat dairy farm without any history of paratuberculosis and randomly allocated to the experimental groups. Animals were housed and raised under appropriate biological containment facilities (BSL-2) located at the institute premises with community pen and straw bedding.

MAP culture and inoculation: Goats were inoculated with the Ejlskov 2007 MAP strain prepared as described for calves above (3.1) and individually dosed three times in 20 ml MAP culture mixed with warm milk replacer at days 4, 7 and 11 after arrival. The dose was reduced 1:5 compared to calves with an estimated total dose of 4×10⁹ live MAP bacilli.

Vaccine composition: Three different forms of the MAP vaccine were tested in formulation with CAF09 adjuvant. CAF09 is a cationic liposome composed of DDA, MMG and Poly I:C combined in a wt/wt ratio of 2500 μg/500 μg/500 μg per dose. Vaccine antigens were diluted appropriately, mixed 1:1 with CAF09 for a total volume of 2 ml/dose and were then allowed to adsorb to the adjuvant CAF09 for 1 h at room temperature before injection. The FadE5 vaccine contained 100 μg recombinantly-expressed MAP latency-associated polypeptide (MAP3694c), the FET11 vaccine contained 40 μg of the MAP1507, MAP1508, MAP3783, MAP3784 fusion polypeptide and 60 μg MAP3694c, and the FET13 vaccine contained 100 μg of a single MAP1507, MAP1508, MAP3783, MAP3784, MAP3694c fusion polypeptide. All polypeptides were produced and purified as described in Example 1.

Vaccination groups and procedure: The goats were randomly assigned to either FadES (n=4), FET11 (n=5), FET13 (n=5) and vaccine control (n=5) groups, respectively. Goats were immunized twice at 14 and 18 weeks post MAP inoculation. Goats were vaccinated by the sub-cutaneous route in the right mid-neck region about 5 cm ahead of the prescapular lymph node. Control goats did not receive any vaccine.

4.2 Immunization with FadE5, FET11 and FET13 Induces Elevated Levels of FadE5-Specific IFN-γ Responses

The immunogenicity of Fet11, FET13 and FadES adjuvanted with CAF09 was examined by measuring antigen-specific IFN-γ responses in whole-blood, sampled from goats at multiple time points up to 32 weeks of age, as shown in FIG. 7. Control (non-vaccinated) goats did not show antigen-specific response to the vaccine protein (all individual samples were below 30 pg/ml). Vaccinated goats responded to immunization with significantly elevated levels of IFN-γ at the first sampled time point 14 days after first vaccination compared to controls. There was a significant response to FadES in all groups irrespective of whether the FadES polypeptide was either the only MAP antigen in the administered vaccine (FadES), or comprised in a single fusion polypeptide with all 5 antigens (FET13); or administered in combination with the MAP1507, MAP1508, MAP3783, MAP3784 fusion polypeptide. These responses are comparable to the response seen in FET11 vaccinated calves and demonstrate that immunization with FadE5 alone or included in a fusion polypeptide is able to induce IFN-γ responses when administered in an appropriate adjuvant.

Example 4.3 FadE5, FET11 and FET13 Immunization Lowers Tissue Colonization of MAP

At 32 weeks post MAP inoculation, the goats were euthanized and necropsied. Fifteen tissue samples from each animal were collected and processed for IS900 qPCR: ileocaecal valve, ileum (0 cm, −25 cm, −50 cm, −75 cm, −100 cm; distance indicated relative to the location of ileocaecal valve in proximal direction), and jejunum (−150 cm, −250 cm, −300 cm), colon (+25 cm, +50 cm distance indicated relative to the location of ileocaecal valve in caudal direction), and four lymph node samples: the ileocaecal LN, the colonic LN and two samples of mesenteric LN draining jejunum at −100 cm and −250 cm proximal to the ileocaecal valve. The tissue samples (8 cm in length) were rinsed with sterile PBS. Epithelium, submucosa, and lamina propria were scraped from the serosa with a sterile object glass and suspended in 5 ml sterile PBS. The tissue scrapings were homogenized by blending in a rotor/stator type tissue homogenizer (Tissue-Tearor from BioSpec Products Inc. 280 North Virginia Avenue, Bartlesville, Okla. 74003 USA). Samples from each animal were processed for DNA extraction, and relative quantification of MAP was performed using qPCR (see Example 2), as shown in FIG. 8. The distal part of jejunum and ileum is recognized as the predilection site of MAP infection and are thus more likely to harbor infection. The FAdES immunized goats were consistently low in MAP numbers at all samples locations while non-immunized controls had one or several samples with much higher MAP numbers in tissues. This was most evident in the predilection site of ileum and distal jejunum and shows that immunization with FadES alone induces a protective immunity to MAP. Vaccination with the all the polypeptides of the invention in the FET11 or FET13 vaccine constructs, provide better protection against MAP than FadES alone with reduced group mean values at all sampling locations.

### Example 5

Immunogenicity of FET11 Vaccine in Different Adjuvants

This study demonstrates that the polypeptides of multistage subunit vaccine of the invention (FET11) can be administered in different adjuvants without compromising the compatibility with serologic surveillance for paratuberculosis or skin test for bovine TB. The vaccines were tested in non-infected calves.

5.1 Preparation of the Vaccine and the Vaccination Protocol

Animals: Twelve male jersey calves with a mean age of eight weeks were obtained from a dairy farm with an active program for control and surveillance for paratuberculosis. Animals were housed and raised under appropriate biological containment facilities (BSL-2) located at the institute premises with community pen and straw bedding.

Vaccine composition: The fusion polypeptide and single polypeptide of the multi-stage vaccine, FET11, were produced and purified as described in Example 1 and formulated in CAF01, CAF09 and Montanide ISA61VG adjuvants. CAF01 is described in Example 3.1. CAF09 is described in Example 4.1. Montanide ISA61VG is a water-in-oil emulsion (Seppic, France). The vaccines contained 20 μg MAP1507, MAP1508, MAP3783, MAP3784 fusion polypeptide+30 μg MAP3694c per calf and adjuvant in the ratio of 1:1 for CAF01 and CAF09 and 1:1^{1/2} for Montanide ISA 61 VG in a 2 ml dose. CAF01 and CAF09 adjuvants were mixed with antigen solution and allowed to absorb for 1 h at room temperature before injection. Montanide was mixed with antigen solution via an i-connector as per manufacturer's instructions.

Vaccination groups and procedure: The calves were randomly assigned to four vaccination groups comprising CAF01, CAF09, Montanide and vaccine control groups, respectively. Calves were vaccinated by the sub-cutaneous route in the right mid-neck region about 7 cm ahead of the prescapular lymph node at nine weeks of age and revaccinated 4 weeks later. Control calves did not receive any vaccine.

5.1 Vaccine Formulation in CAF01, CAF09 or Montanide ISA 61 VG does not Interfere with Single Intradermal Comparative Cervical Tuberculin Testing

All 12 animals were subjected to SICCT at 4 weeks post second vaccination. All animals were SICCT test negative as no increase in skin thickness (in mm) was measured 72 hours after intradermal injection of PPDb and PPDa in any of the 12 animals. Animals were thus also negative when evaluated only for response to PPDb. The results show that the polypeptides of the invention do not induce reactivity in SICCT test for bovine TB irrespective of adjuvant formulation.

5.2 Vaccine Formulation in CAF01, CAF09 or Montanide ISA 61 VG does not Interfere with Serological Surveillance for Paratuberculosis by ID Screen Paratuberculosis ELISA

All 12 animals were tested in the ID Screen Paratuberculosis ELISA at 3½ weeks post second vaccination (FIG. 9). Serum levels of IgG antibodies directed against MAP was expressed as S/P percentage (cut off S/P % 70%) and samples from all the 12 animals was found to be negative (range of S/P/%: −1.36 to 17.76 and mean value: 2.33). The results show that the polypeptides of the invention do not induce reactivity in a commercial test for antibody-based surveillance of paratuberculosis irrespective of adjuvant formulation.

### REFERENCES

1. Aagaard, C., T. Hoang, J. Dietrich, P. J. Cardona, A. Izzo, G. Dolganov, G. K. Schoolnik, J. P. Cassidy, R. Billeskov, and P. Andersen. 2011. A multistage tuberculosis vaccine that confers efficient protection before and after exposure. Nat. Med. 17:189-194.
2. Agger, E. M., I. Rosenkrands, J. Hansen, K. Brahimi, B. S. Vandahl, C. Aagaard, K. Werninghaus, C. Kirschning, R. Lang, D. Christensen, M. Theisen, F. Follmann, and P. Andersen. 2008. Cationic liposomes formulated with synthetic mycobacterial cordfactor (CAF01): a versatile adjuvant for vaccines with different immunological requirements. PLoS One 3:e3116.
3. Brandt, L., M. Elhay, I. Rosenkrands, E. B. Lindblad, and P. Andersen. 2000. ESAT-6 subunit vaccination against Mycobacterium tuberculosis. Infect. Immun. 68:791-795.
4. Buergelt, C. D., C. Hall, K. McEntee, and J. R. Duncan. 1978. Pathological evaluation of paratuberculosis in naturally infected cattle. Vet. Pathol. 15:196-207.
5. Bustin, S. A., V. Benes, J. A. Garson, J. Hellemans, J. Huggett, M. Kubista, R. Mueller, T. Nolan, M. W. Pfaffi, G. L. Shipley, J. Vandesompele, and C. T. Wittwer. 2009. The MIQE guidelines: minimum information for publication of quantitative real-time PCR experiments. Clin. Chem. 55:611-622.
6. Chen, J. W., S. M. Faisal, S. Chandra, S. P. McDonough, M. A. Moreira, J. Scaria, C. F. Chang, J. P. Bannantine, B. Akey, and Y. F. Chang. 2012. Immunogenicity and protective efficacy of the Mycobacterium avium subsp. paratuberculosis attenuated mutants against challenge in a mouse model. Vaccine 30:3015-3025.
7. Decaro, N., F. Amorisco, C. Desario, E. Lorusso, M. Camero, A. L. Bellacicco, R. Sciarretta, M. S. Lucente, V. Martella, and C. Buonavoglia. 2010. Development and validation of a real-time PCR assay for specific and sensitive detection of canid herpesvirus I. J. Virol. Methods 169:176-180.
8. Eltholth, M. M., V. R. Marsh, W. S. Van, and F. J. Guitian. 2009. Contamination of food products with Mycobacterium avium paratuberculosis: a systematic review. J. Appl. Microbiol. 107:1061-1071.
9. Flynn, J. L., J. Chan, K. J. Triebold, D. K. Dalton, T. A. Stewart, and B. R. Bloom. 1993. An essential role for interferon gamma in resistance to Mycobacterium tuberculosis infection. J. Exp. Med. 178:2249-2254.
10. Green, E. P., M. L. Tizard, M. T. Moss, J. Thompson, D. J. Winterbourne, J. J. McFadden, and J. Hermon-Taylor. 1989. Sequence and characteristics of IS900, an insertion element identified in a human Crohn's disease isolate of Mycobacterium paratuberculosis. Nucleic Acids Res. 17:9063-9073.
11. Hines, M. E., J. R. Stabel, R. W. Sweeney, F. Griffin, A. M. Talaat, D. Bakker, G. Benedictus, W. C. Davis, G. W. de Lisle, I. A. Gardner, R. A. Juste, V. Kapur, A. Koets, J. McNair, G. Pruitt, and R. H. Whitlock. 2007. Experimental challenge models for Johne's disease: a review and proposed international guidelines. Vet. Microbiol. 122:197-222.
12. Huntley, J. F., J. R. Stabel, M. L. Paustian, T. A. Reinhardt, and J. P. Bannantine. 2005. Expression library immunization confers protection against Mycobacterium avium subsp. paratuberculosis infection. Infect. Immun. 73:6877-6884.
13. Jungersen, G., A. Huda, J. J. Hansen, and P. Lind. 2002. Interpretation of the gamma interferon test for diagnosis of subclinical paratuberculosis in cattle. Clin. Diagn. Lab Immunol. 9:453-460.
14. Kalis, C. H., J. W. Hesselink, H. W. Barkema, and M. T. Collins. 2001. Use of long-term vaccination with a killed vaccine to prevent fecal shedding of Mycobacterium avium subsp paratuberculosis in dairy herds. Am. J. Vet. Res. 62:270-274.
15. Kathaperumal, K., S. U. Park, S. McDonough, S. Stehman, B. Akey, J. Huntley, S. Wong, C. F. Chang, and Y. F. Chang. 2008. Vaccination with recombinant Mycobacterium avium subsp. paratuberculosis proteins induces differential immune responses and protects calves against infection by oral challenge. Vaccine 26:1652-1663.
16. Kawaji, S., D. L. Taylor, Y. Mori, and R. J. Whittington. 2007. Detection of Mycobacterium avium subsp. paratuberculosis in ovine faeces by direct quantitative PCR has similar or greater sensitivity compared to radiometric culture. Vet. Microbiol. 125:36-48.
17. Koets, A., A. Hoek, M. Langelaar, M. Overdijk, W. Santema, P. Franken, W. Eden, and V. Rutten. 2006. Mycobacterial 70 kD heat-shock protein is an effective subunit vaccine against bovine paratuberculosis. Vaccine 24:2550-2559.
18. Kulberg, S., P. Boysen, and A. K. Storset. 2004. Reference values for relative numbers of natural killer cells in cattle blood. Dev. Comp Immunol. 28:941-948.
19. Lindenstrom, T., E. M. Agger, K. S. Korsholm, P. A. Darrah, C. Aagaard, R. A. Seder, I. Rosenkrands, and P. Andersen. 2009. Tuberculosis subunit vaccination provides long-term protective immunity characterized by multifunctional CD4 memory T cells. J. Immunol. 182:8047-8055.
20. Mikkelsen, H., C. Aagaard, S. S. Nielsen, and G. Jungersen. 2012. Correlation of antigen-specific IFN-gamma responses of fresh blood samples from Mycobacterium avium subsp. paratuberculosis infected heifers with responses of day-old samples co-cultured with IL-12 or anti-IL-10 antibodies. Vet. Immunol. Immunopathol. 147:69-76.
21. Mikkelsen, H., C. Aagaard, S. S. Nielsen, and G. Jungersen. 2011. Novel antigens for detection of cell mediated immune responses to Mycobacterium avium subsp. paratuberculosis infection in cattle. Vet. Immunol. Immunopathol. 143:46-54.
22. Mikkelsen, H., G. Jungersen, and S. S. Nielsen. 2009. Association between milk antibody and interferon-gamma responses in cattle from Mycobacterium avium subsp. paratuberculosis infected herds. Vet. Immunol. Immunopathol. 127:235-241.
23. Morrison, W. I., F. J. Bourne, D. R. Cox, C. A. Donnelly, G. Gettinby, J. P. McInerney, and R. Woodroffe. 2000. Pathogenesis and diagnosis of infections with Mycobacterium bovis in cattle. Independent Scientific Group on Cattle TB. Vet. Rec. 146:236-242.
24. Olsen, I., P. Boysen, S. Kulberg, J. C. Hope, G. Jungersen, and A. K. Storset. 2005. Bovine NK cells can produce gamma interferon in response to the secreted mycobacterial proteins ESAT-6 and MPP14 but not in response to MPB70. Infect. Immun. 73:5628-5635.
25. Olsen, I., S. Tollefsen, C. Aagaard, L. J. Reitan, J. P. Bannantine, P. Andersen, L. M. Sollid, and K. E. Lundin. 2009. Isolation of Mycobacterium avium subspecies paratuberculosis reactive CD4 T cells from intestinal biopsies of Crohn's disease patients. PLoS. One. 4:e5641.
26. Ott, S. L., S. J. Wells, and B. A. Wagner. 1999. Herd-level economic losses associated with Johne's disease on US dairy operations. Prey. Vet. Med. 40:179-192.
27. Pathak, S., J. A. Awuh, N. A. Leversen, T. H. Flo, and B. Asjo. 2012. Counting mycobacteria in infected human cells and mouse tissue: a comparison between qPCR and CFU. PLoS. One. 7:e34931.
28. Price, S. J. and J. C. Hope. 2009. Enhanced secretion of interferon-gamma by bovine gammadelta T cells induced by coculture with Mycobacterium bovis-infected dendritic cells: evidence for reciprocal activating signals. Immunology 126:201-208.
29. Rosseels, V. and K. Huygen. 2008. Vaccination against paratuberculosis. Expert. Rev. Vaccines. 7:817-832.
30. Roussel, Y., A. Harris, M. H. Lee, and M. Wilks. 2007. Novel methods of quantitative real-time PCR data analysis in a murine Helicobacter pylori vaccine model. Vaccine 25:2919-2929.
31. Rozen, S. and H. Skaletsky. 2000. Primer3 on the WWW for general users and for biologist programmers. Methods Mol. Biol. 132:365-386.
32. Santema, W., S. Hensen, V. Rutten, and A. Koets. 2009. Heat shock protein 70 subunit vaccination against bovine paratuberculosis does not interfere with current immunodiagnostic assays for bovine tuberculosis. Vaccine 27:2312-2319.
33. Santema, W. J., J. Poot, R. P. Segers, D. J. Van den Hoff, V. P. Rutten, and A. P. Koets. 2012. Early infection dynamics after experimental challenge with Mycobacterium avium subspecies paratuberculosis in calves reveal limited calf-to-calf transmission and no impact of Hsp70 vaccination. Vaccine .
34. Sechi, L. A., L. Mara, P. Cappai, R. Frothingam, S. Ortu, A. Leoni, N. Ahmed, and S. Zanetti. 2006. Immunization with DNA vaccines encoding different mycobacterial antigens elicits a Th1 type immune response in lambs and protects against Mycobacterium avium subspecies paratuberculosis infection. Vaccine 24:229-235.
35. Stabel, J. R. 2000. Transitions in immune responses to Mycobacterium paratuberculosis. Vet. Microbiol. 77:465-473.
36. Stabel, J. R. and S. Robbe-Austerman. 2011. Early immune markers associated with Mycobacterium avium subsp. paratuberculosis infection in a neonatal calf model. Clin. Vaccine Immunol. 18:393-405.
37. Sweeney, R. W. 2011. Pathogenesis of paratuberculosis. Vet. Clin. North Am. Food Anim Pract. 27:537-46, v.
38. Weinrich, O. A., L. A. van Pinxteren, O. L. Meng, R. P. Birk, and P. Andersen. 2001. Protection of mice with a tuberculosis subunit vaccine based on a fusion protein of antigen 85b and esat-6. Infect. Immun. 69:2773-2778.
39. Whelan, A. O., B. Villarreal-Ramos, H. M. Vordermeier, and P. J. Hogarth. 2011. Development of an Antibody to Bovine IL-2 Reveals Multifunctional CD4 T(EM) Cells in Cattle Naturally Infected with Bovine Tuberculosis. PLoS. One. 6:e29194.
40. Whitlock, R. H. and C. Buergelt. 1996. Preclinical and clinical manifestations of paratuberculosis (including pathology). Vet. Clin. North Am. Food Anim. Pract. 12:345-356.
41. Windsor, P. A. and R. J. Whittington. 2010. Evidence for age susceptibility of cattle to Johne's disease. Vet. J. 184:37-44.
42. P Ryan, 1 M W et al., PCR detection of Mycobacterium paratuberculosis in Crohn's disease granulomas isolated by laser capture microdissection Gut. 2002 November; 51(5): 665-670.

43. Naser SA et al. Lancet 2004, 364:1039-44
44. Bull T J et al. J. Cin Microbiol. 2003, 41:2915-23
45. Greenberg G R. Antibiotics should be used as first-line therapy for Crohn's disease. Inflamm Bowel Dis.2004; 10(3):318-320.
46. Olsen I, et al., PLoS One. 2009 May 22; 4(5):e5641. Isolation of Mycobacterium avium subspecies paratuberculosis reactive CD4 T cells from intestinal biopsies of Crohn's disease patients.
47. Hugot J P, et al., Association of NOD2 leucine-rich repeat variants with susceptibility to Crohn's disease., Nature 2001 May 31;411(6837):599-603

48. Radford-Smith G, et al., (2006) Associations between NOD2/CARD15 genotype and phenotype in Crohn's disease—Are we there yet?. World J. Gastroenterol. 12(44):7097-103.
49. Barrett, Jeffrey C et al., (2008). “Genome-wide association defines more than 30 distinct susceptibility loci for Crohn's disease”. Nature Genetics 40(8):955-62.
50. Kathaperumal, K., V. Kumanan, S. McDonough, L. H. Chen, S. U. Park, M. Moreira, B. Akey, J. Huntley, C. F. Chang, and Y. F. Chang. 2009. Evaluation of immune responses and protective efficacy in a goat model following immunization with a coctail of recombinant antigens and a polyprotein of Mycobacterium avium subsp. paratuberculosis. Vaccine 27:123-135.
51. Lu, Z., Y. H. Schukken, R. L. Smith, R. M. Mitchell, Y. T. Grohn. 2013. Impact of imperfect Mycobacterium avium subsp. paratuberculosis vaccines in dairy herds: A mathematical modeling approach. Preventive Veterinary Medicine 108: 148-158.
52. Weigoldt, M., J. Meens, F. C. Bange, A. Pich, G. F. Gerlach, R. Goethe. 2013. Metabolic adaptation of Mycobacterium avium subsp. paratuberculosis to the gut environment. Microbiology 159: 380-391.

1.-20. (canceled)

21. An immunogenic polypeptide for use as a preventive and therapeutic vaccine against a Mycobacterial infection in a human or an animal, wherein said polypeptide comprises an amino acid sequence of SEQ ID NO: 2.

22. The immunogenic polypeptide for use as a preventive and therapeutic vaccine against a Mycobacterial infection in a human or an animal according to claim 21, wherein administration of said polypeptide provides protective immunity in both an infected human or animal and in an uninfected human or animal.

23. The immunogenic polypeptide for use as a preventive and therapeutic vaccine against a Mycobacterial infection in a human or an animal according to claim 22, wherein administration of said polypeptide provides protective immunity in an infected human or animal by decreasing mycobacterial numbers and preventing reactivation of the mycobacterial infection.

24. The immunogenic polypeptide for use as a preventive and therapeutic vaccine against a Mycobacterial infection in a human or an animal according to claim 21, wherein said polypeptide is combined with at least one additional immunogenic polypeptide, wherein each additional polypeptide has an amino acid sequence selected from the group consisting of SEQ ID NOs: 4, 6, 8 and 10; or an amino acid sequence having at least 85% amino acid sequence identity to one of SEQ ID NOs: 4, 6, 8 and 10.

25. The immunogenic polypeptide for use as a preventive and therapeutic vaccine against a Mycobacterial infection in a human or an animal according to claim 24, wherein at least two of said polypeptides is comprised within a fusion polypeptide.

26. A vaccine for use in prevention and treatment of a Mycobacterium avium subsp. paratuberculosis infection, wherein said vaccine comprises:
a. the immunogenic polypeptide having SEQ ID NO: 2 of claim 21; or
b. the immunogenic polypeptide of (a) combined with at least one additional immunogenic polypeptide, wherein said additional polypeptide has at least 85% amino acid sequence identity to a sequence selected from the group consisting of SEQ ID NOs: 4, 6, 8, and 10; or
c. the immunogenic polypeptides of (b), wherein at least two of said polypeptides is comprised within a fusion polypeptide; or
d. one or more nucleic acid molecule encoding the immunogenic polypeptide(s) of (a), (b) or (c).

27. The vaccine for use according to claim 26, wherein administration of said vaccine provides protective immunity in both an uninfected human or animal and an infected human or animal.

28. The vaccine for use according to claim 26, wherein administration of said vaccine provides protective immunity in an infected human or animal by decreasing mycobacterial numbers and preventing reactivation of the mycobacterial infection.

29. The vaccine for use according to claim 26, comprising a first polypeptide having an amino acid sequence of SEQ ID No: 14 and a fusion polypeptide having at least 85% amino acid sequence identity to SEQ ID No: 12.

30. The vaccine for use according to claim 26, comprising a fusion polypeptide having at least 85% amino acid sequence identity to SEQ ID No: 18.

31. The vaccine for use according to claim 26, wherein the first polypeptide comprises the amino acid sequence of SEQ ID NO: 2, and where the at least one additional immunogenic polypeptide has an amino acid sequence selected from the group consisting of SEQ ID NOs: 4, 6, 8 and 10.

32. The vaccine for use according to claim 26, wherein the vaccine is for prevention and treatment of a Mycobacterium avium subsp. paratuberculosis infection in a mammal

33. The vaccine for use according to claim 26, wherein the vaccine is for prevention and treatment of Crohn's disease in a human.

34. The vaccine for use according to claim 31, for prevention and treatment of paratuberculosis in a mammal selected from the group consisting of a porcine, ruminant, equine, feline, canine, primate, and rodent.

35. The vaccine for use according to claim 26, further comprising a pharmaceutically acceptable carrier, adjuvant and/or immunomodulator.

36. The vaccine for use according to claim 26, for administration to a mammal by saline or buffered saline injection of naked DNA or RNA or injection of DNA plasmid or linear gene expressing DNA fragments coupled to particles, inoculated by gene gun or delivered by a viral or bacterial vector.

37. The vaccine for use according to claim 26, wherein said one or more nucleic acid molecule is incorporated in the genome of a self-replicating non-pathogenic recombinant carrier, and wherein said carrier is capable of in vivo expression of said immunogenic polypeptides encoded by said more or more nucleic acid molecule.

38. The vaccine of claim 26, wherein said one or more nucleic acid molecule comprises a nucleic acid sequence selected from the group consisting of SEQ ID NOs: 1, 3, 5, 7, 9, 11, 13 and 17.

39. The vaccine for use according to claim 26, for administration by parenteral injection.

40. A method of preparing the vaccine of claim 26, comprising the steps of:
a. synthesizing an immunogenic polypeptide having amino acid sequence SEQ ID NO: 2, or synthesizing the immunogenic polypeptide combined with at least one additional immunogenic polypeptide wherein said additional polypeptide has at least 85% sequence identity to a sequence selected from the group consisting of SEQ ID NOs: 4, 6, 8, and 10;
solubilizing or dispersing the polypeptide(s) in an aqueous medium, and optionally adding a pharmaceutically acceptable adjuvant.
